(19) United States
(12) Patent Application Publication
(10) Pub. No.: US 2023/0390167 A1
(43) Pub. Date: Dec. 7, 2023

(54) PROCESS FOR PRODUCING MICROPARTICLES LADEN WITH AN AROMA CHEMICAL

(71) Applicant: BASF SE, Ludwigshafen am Rhein (DE)

(72) Inventors: Bernd Dieter OSCHMANN, Ludwigshafen am Rhein (DE); Wolfgang KRAUSE, Lampertheim (DE); Michael GOLLNER, Ludwigshafen am Rhein (DE)

(21) Appl. No.: 18/033,087

(22) PCT Filed: Oct. 29, 2021
(86) PCT No.: PCT/EP2021/080114

§ 371 Date: Apr. 21, 2023

(30) Foreign Application Priority Data
Oct. 30, 2020 (EP) 20205006.8

(51) Int. Cl. A61K 8/11; A61Q 13/00; A61K 8/85; B01J 13/08; C11D 3/50

(52) U.S. Cl. CPC A61K 8/11; A61Q 13/00; A61K 8/85; B01J 13/08; C11D 3/505; A61K 2800/412; A61K 2800/22

(57) ABSTRACT

The present invention relates to a process for producing polymer microparticles laden with at least one aroma chemical, which comprises i. dissolving the at least one aroma chemical and an organic polymer composition, which contains at least one polyester as a main constituent, in a volatile organic solvent having a boiling point lower than the boiling point of the aroma chemical and having a solubility in water of at most 100 g/L at 20° C. and 1013 mbar, whereby a solution of the aroma chemical and the organic polymer composition is obtained; ii. emulsifying the solution obtained in step i. in an aqueous medium containing at least one dispersant; iii. removing the volatile organic solvent from the emulsion by evaporation at a temperature of below 80° C. and at a pressure below atmospheric pressure, whereby an aqueous suspension of the laden polymer microparticles is obtained. The present invention also relates to microparticles laden with an aroma chemical, obtainable by the process described herein and to products containing the microparticle composition. The compositions are useful as additives for imparting a scent or flavour to a product. The compositions allow for controlled release of the aroma chemicals.

The present invention relates to processes for producing microparticles laden with at least one aroma chemical, especially to a process for producing biodegradable microparticles laden with at least one aroma chemical by way of in situ-encapsulation using an emulsion-solvent evaporation process. The invention also relates to compositions of microparticles laden with an aroma chemical and to the use thereof.

Microcapsules have various uses as carriers for active substances, for example for crop protecting agents, pharmaceutical agents, fragrances and aromas, but also for reactive substances or catalysts for industrial applications. They typically comprise a polymer material that envelops the material to be encapsulated. Advantages of a formulation of this kind are in particular:
protection of reactive actives from environmental effects;
safe and practical handling of toxic or unstable actives;
controlled release of actives;
prevention of premature mixing of substances;
the handling of liquid actives as solids.

An overview of the methodology of microencapsulation of actives can be found in H. Mollet, A. Grubenmann, “Formulation Technology”, chapter 6.4 (Microencapsulation), Wiley VCH Verlag GmbH, Weinheim 2001, and the literature cited therein.

In many consumer products, it is desirable for aroma chemicals, especially perfume raw materials, to be released slowly over time. For example in case of perfume materials, the more volatile perfume raw materials are responsible for the “fresh feeling” that consumers experience. Therefore, it is desirable for the more volatile perfume raw materials to be released in a slow, controlled manner.

One frequently used approach to the controlled release of fragrances is their microencapsulation. Typically, these microcapsules comprise a hydrophobic liquid core containing the aroma chemical which is surrounded by a polymer shell. Such polymers may be, for example, a polyurethane, polyurea, polyamide, polyester, polycarbonate, urea/formaldehyde resin, a melamine/formaldehyde resin, a polystyrene, or an acrylate polymer. The microparticles are typically prepared by an interfacial polymerization of the respective monomers or oligomers, which form the polymer shell in an oil-in-water (o/w) emulsion of the aroma chemical (see e.g. WO 2008/066773 and WO 2009/090169 and the references cited therein).

EP 2233557 describes a process for preparing a perfume encapsulate for use in laundry detergent compositions. The process comprises emulsifying a hydrophobic perfume-copolymer complex in water to form an oil-in-water emulsion; at least partially dissolving an encapsulation material, such as maltodextrin, in water, removing at least some water from the oil-in-water emulsion comprising the hydrophobic perfume-polymer complex and the encapsulating material to form an encapsulate. The encapsulate is said to have good dissolution profiles, whilst retaining good thermal stability profiles. The process has several disadvantages, as it is applicable only to those perfume compounds, which are capable to form a complex with the polymer. Moreover, multiple steps are required to produce the microparticles.

WO 2018/130433 suggests swellable silica microparticles with a nonionic polysaccharide deposition aid attached to their outer surface for encapsulating perfumes. The microparticles have a polymer shell typically made of an aminoplast polycondensate, such as melamine formaldehyde polycondensate. The process requires several steps including the preparation of the swellable silica microparticles, soaking them with the perfume and encapsulating them with a polymer.

However, consumers are becoming increasingly concerned about the presence of microplastics in their home and personal care products. Therefore, there is a demand for providing delivery forms for aroma chemicals which can be produced with reduced amounts of non-degradable plastic material.

WO 2018/065481 and WO 2019/193094 disclose a process for preparing microparticles laden with aroma chemicals. The process comprises producing porous microparticles made of a thermoplastic biodegradable polyester material and suspending the microparticles in a liquid aroma chemical or solution of the aroma chemical. The porous microparticles are produced by providing an w/o-emulsion of the polyester material dissolved in a water-immiscible solvent as a continuous phase and an aqueous solution of a pore-forming agent as the discontinuous phase, emulsifying the w/o-emulsion in water to obtain a water-in-oil-in-water (w/o/w) emulsion and removing the organic solvent by evaporation. In order to achieve controlled release, the microparticles laden with the aroma chemical must be closed or sealed, which is effected by heating the laden microparticles over a time-prolonged period, which can lead to degradation of the active and to unwanted agglomeration or even destruction of the laden microparticles. Moreover, the release characteristics are not always satisfactory. Moreover, several steps have to be carried out.

A similar process is described in WO 2020/089191, where porous microparticles laden with an aroma chemical are coated or where the porous microparticles laden with an aroma chemical in a liquid matrix which is solidified. Thereby, the time consuming heat treatment can be avoided. On the other hand, the relative amount of aroma chemical is comparatively low due to the required matrix or coating.

The aforementioned production methods for microparticles laden with an aroma chemical require either prolonged heating of the laden microparticles which may be detrimental to the microparticles and/or the aroma chemical or the presence of a coating or matrix. Also, the preparation of such particles is carried out in separate steps, one being the preparation of hollow microparticles, followed by loading with desired substances and prolonged heating to prevent premature exit of the substance. Thus, alternative processes are required to simplify the production process.

PCT/EP2020/069538 provides an improved process for sealing porous microparticles laden with aroma chemicals. The process comprises producing porous microparticles from a polyester material by the methods disclosed in WO 2018/065481 and WO 2019/193094 followed by loading the microparticles with aroma chemical and then short-time heating the microparticles to a temperature above the softening temperature of the polyester material.

It is therefore an object of the invention to provide a simple and straightforward process for preparing microparticles laden with at least one aroma chemical which do not require prolonged heating during the process or the presence of a matrix or coating. The microparticles are to have a relatively low surface porosity causing slow release properties. The laden microparticles are to be producible in a simple process and with high yield but without significant agglomeration or even destruction of the laden microparticles.

It has been found that, surprisingly, these and further objects are achieved by the process described hereinafter and the aroma chemical-filled microparticles that are obtainable thereby.

The present invention therefore relates to a process for producing polymer microparticles laden with at least one aroma chemical, which comprises
i. dissolving the at least one aroma chemical and an organic polymer composition, which contains at least one polyester as a main constituent, in a volatile organic solvent having a boiling point lower than the boiling point of the aroma chemical and having a solubility in water of at most 100 g/L, e.g. 1 mg/L to 100 g/L at 20° C. and 1013 mbar, whereby a solution of the aroma chemical and the organic polymer composition is obtained;
ii. emulsifying the solution obtained in step i. in an aqueous medium containing at least one dispersant;
iii. removing the volatile organic solvent from the emulsion by evaporation at a temperature of below 80° C. and at a pressure below atmospheric pressure, whereby an aqueous suspension of the laden polymer microparticles is obtained,

wherein the concentration of the organic polymer composition in the solution of step i. is in the range of 1 to 250 g/kg, based on the total weight of the solution; and the weight ratio of the aroma chemical to organic polymer composition is in the range of 1:50 to 2:1.

The present invention further relates to a composition of microparticles laden with aroma chemical, which is obtainable by a process of the invention, and to the use thereof, especially in a product selected from perfumes, washing and cleaning products, cosmetic products, personal care products, hygiene articles, foods, food supplements, fragrance dispensers and fragrances.

The present invention further relates to products comprising an inventive composition of microparticles laden with aroma chemical.

The invention is associated with a number of advantages:
The process of the present invention produces microparticles laden with an aroma chemical which allow for controlled release of the aroma chemical, namely by burst release caused by mechanical pressure, by diffusion or by degradation of the biodegradable membrane which leads to slow release effects desirable for certain applications.
The process of the present invention is much simpler, more straightforward and less problematic than comparable processes requiring separate steps of producing hollow particles, followed by filling the microparticles with an aroma chemical and finally sealing the porous surface of the pores e.g. by applying heat or a coating.
By the process of the invention, the surface porosity of the microparticles laden with the aroma chemical is greatly reduced without significantly destroying or agglomerating the laden microparticles due to extended exposure to heat.
As the surface porosity of the laden microparticles is reduced, they can be stored over a prolonged period without any significant loss of the active, which is in particular important in case of sensible or volatile organic actives.

The present invention relates more particularly to the following embodiments 1 to 33:
1. A process for producing microparticles laden with at least one aroma chemical, wherein the above described steps i., ii. and iii. are successively carried out.
2. The process according to embodiment 1, where the organic solvent has a boiling point at 1013 mbar in the range of 35 to 85° C., in particular in the range of 38 to 80° C.
3. The process according to the preceding embodiment, where the organic solvent is selected from the group consisting of dichloromethane, trichloromethane, ethyl acetate, benzene, n-hexane, cyclohexane, n-pentane, diethyl ether, methyl tert.-butyl ether, diisopropyl ether and mixtures thereof with 2-butanone.
4. The process according to any of the two preceding embodiments, where the organic solvent contains dichloromethane as a main constituent.
5. The process according to any of the preceding embodiments, where the concentration of the organic polymer composition in the solution of step i. is in the range of 2 to 150 g/kg, especially 3 to 100 g/kg, based on the total weight of the solution.
6. The process according to any of the preceding embodiments, where the weight ratio of the aroma chemical to polymer is in the range of 1:20 to 1.5:1 or 1:20 to 1:1 or 1:20 to 1:1.5 and especially in the range of 1:10 to 1:1 or 1:10 to 1:2.
7. The process according to any of the preceding embodiments, where the dispersant contained in the aqueous phase is selected from the group consisting of polysaccharides, polyvinyl alcohols, polymers bearing sulfonate groups, polyvinylpyrolidone copolymers of vinylpyrrolidone and inorganic pickering stabilizers.
8. The process according to any of the preceding embodiments, where the dispersant comprises a polyvinyl alcohol, in particular a polyvinyl alcohol having a degree of hydrolysis in the range of 70 to 99.9%.
9. The process according to any of the preceding embodiments, where in step ii. the relative weight of the solution obtained in step i. and the aqueous medium is in the range of 1:5 to 1:1, in particular in the range of 1:3 to 1:1 and especially in the range of 1:2.5 to 1:1 or 1:2 to 1:1.
10. The process according to any of the preceding embodiments, where the amount of dispersant is in the range of 0.1 to 10% by weight, in particular in the range of 0.2 to 5% by weight, based on the total weight of the solution obtained in step i.
11. The process according to any of the preceding embodiments, where the amount of dispersant is in the range of 2 to 30% by weight, in particular in the range of 3 to 20% by weight, based on the total weight of the polymer composition and the aroma chemical contained in the solution obtained in step i.
12. The process according to any of the preceding embodiments, where the concentration of the dispersant in the aqueous phase is in the range of 0.1 to 5% by weight, in particular in the range of 0.3 to 2% by weight, based on the total weight of the aqueous phase.
13. The process according to any of the preceding embodiments, where emulsification comprises mixing the solution of step i. with the aqueous phase and homogenization of the mixture.
14. The process according to embodiment 13, wherein the homogenization is achieved by one of the following measures or by a combination thereof:
   treatment of the mixture with a rotor stator mixer, in particular a toothed-rim mixer;
   applying ultrasound to the mixture;
   treatment of the mixture with a dispersing disc or a cross-blade stirrer with one or multiple stages;
   passing the mixture through a microfluidic device;
   high pressure mixing.
15. The process according to any one of embodiments 10 or 11, wherein the homogenization is carried out until the average droplet size of the emulsion is at morst or below 400 μm, in particular at most 300 μm, especially at most 250 μm.
16. The process according to any one of the preceding embodiments, where during the evaporation of the solvent the emulsion is agitated.
17. The process according to embodiment 16, where the evaporation of the solvent is carried out at a temperature in the range of 30 to 80° C., in particular in the range of 35 to 60° C. and at a pressure in the range of 20 to 800 mbar, in particular in the range of 50 to 500 mbar.
18. The process according to any one of the preceding embodiments, where the evaporation of the solvent is carried out until the concentration of organic solvent in the aqueous suspension is less than 0.5% by weight, based on the total weight of the suspension.
19. The process according to any one of the preceding embodiments, where the evaporation of the solvent is carried out until the concentration of the microparticles in the aqueous suspension is in the range of 2 to 30% by weight, based on the total weight of the suspension.
20. The process according to any of the preceding embodiments, wherein the polymer composition has a solubility in dichloromethane of at least 50 g/L at 25° C. and 1013 mbar.
21. The process according to any of the preceding embodiments, wherein the polymer composition comprises or essentially consists of at least one aliphatic polyester or comprises or essentially consists of at least one semi-aromatic polyester or comprises or essentially consists of a combination of at least one semi-aromatic polyester with at least one thermoplastic polymer which is not an aliphatic-aromatic polyester.
22. The process according to embodiment 21, wherein the polymer composition, besides the aliphatic-aromatic polyester, additionally comprises at least one further polymer which is different from semi-aromatic polyesters and which is in particular selected from the group consisting of aliphatic polyesters, polyanhydrides, polyesteramides, modified polysaccharides and proteins and mixtures thereof.
23. The process according to any one of the preceding embodiments, wherein the aroma chemical is liquid at 22° C. and 1013 mbar.
24. The process according to any one of the preceding embodiments, wherein the aroma chemical has a boiling point of at least 90° C., in particular at least 120° C. at 1013 mbar, e.g. in the range of 90° C. to 300° C., in particular 120 to 250° C.
25. The process according to any one of the preceding embodiments, which further comprises a step, where the microparticles are isolated from aqueous suspension of the laden polymer microparticles obtained in step iii.
26. A composition of microparticles laden with an aroma chemical, obtainable by a process according to any of the preceding embodiments.
27. The composition according to embodiment 26, which is an aqueous dispersion of microparticles.
28. The composition according to any one of embodiments 26 or 27, which is a powder.
29. The composition according to any one of embodiments 26, 27 or 28, which has at least one of the following features a), b) or c)
   a) the weight ratio of the aroma chemical to polymer in the microparticle is in the range of 1:20 to 1.5:1 or 1:20 to 1:1 or 1:20 to 1:1.5 and especially in the range of 1:10 to 1:1 or 1:10 to 1:2;
   b) the amount of dispersant is in the range of 2 to 30% by weight, in particular in the range of 3 to 20% by weight, based on the total weight of the microparticles;
   c) the microparticles have an average particle diameter D[4,3] in the range of 0.5 to 400 μm, in particular 1 to 300 μm and especially 2 to 200 μm, as determined by static laser light scattering according to ISO 13320:2009.
30. A product comprising a composition according to any of embodiments 26 to 29 in a proportion by weight of 0.01% to 80% by weight based on the total weight of the product.
31. The product of embodiment 30, which is selected from perfumes, washing and cleaning products, cosmetic products, personal care products, hygiene articles, foods, food supplements, fragrance dispensers and fragrances.
32. The use of the composition according to any of embodiments 26 to 29 as an additive for imparting a scent or a flavour to a product, which is in particular selected from perfumes, washing and cleaning products, cosmetic products, personal care products, hygiene articles, foods, food supplements, fragrance dispensers and fragrances.
33. The use of the composition according to any of embodiments 26 to 29 for controlled release of aroma chemicals.

Here and throughout the specification the term “main constituent” with respect to component of a composition means that the relative amount of the component in the composition is higher than any other relevant amount of any other component in said composition. In particular, the term “main constituent” means that the relative amount of the component is at least 50% by weight, in particular least 60% by weight, especially at least 70% by weight or at least 80% by weight, and may be up to 100% by weight, based on the total weight of the respective composition.

Here and throughout the specification the term “essentially consisting of” with respect to component of a composition means that the relative amount of the component is at least 90% by weight, in particular at least 95% by weight and especially at least 99% by weight or 100% by weight, based on the total weight of the composition.

The “molecular weight Mn” or the “molar mass Mn” is the number-average molecular weight or molar mass. The “molecular weight Mw” or the “molar mass Mw” is the mass-average molecular weight or molar mass. “Polydispersity” is as the ratio of weight-average to number-average, i.e. the quotient Mw/Mn.

Unless stated otherwise, the term “room temperature” indicates a temperature of 22° C.

The term “aroma chemical” is understood by the person skilled in the art to mean organic compounds usable as “odorant” and/or as “flavoring”. In the context of the present invention, “odorant” is understood to mean natural or synthetic substances having intrinsic odor. In the context of the present invention, “flavoring” is understood to mean natural or synthetic substances having intrinsic flavor. In the context of the present invention, “odor” or “olfactory perception” is the interpretation of the sensory stimuli which are sent from the chemoreceptors in the nose or other olfactory organs to the brain of a living being. The odor can be a result of sensory perception of the nose of odorant, which occurs during inhalation. In this case, the air serves as odor carrier.

The term “microparticles” means that the particles have dimensions in the micro-meter range, i.e. below 1000 μm, particularly below 800 μm and especially below 600 μm. The value reported here is that value that exceeds 90% by volume of the particles present in a sample, which is also referred to as the D[v, 0.9] value. Typically, at least 90% by volume of the microparticles laden with the aroma chemical have dimensions of at least 1 μm, particularly at least 2 μm and especially at least 5 μm (called the D[v, 0.1] value).

Here and hereinafter, all figures for particle sizes, particle diameters and particle size distributions, including the D[v, 0.1], D[v, 0.5], D[v, 0.9], D[4,3] and D[3,2] values, are based on the distributions ascertained by static laser light scattering to ISO 13320:2009 on samples of the microparticles. The abbreviation SLS is also used hereinafter for the expression “static laser light scattering to ISO 13320:2009”.

In this connection, the D[v, 0.1] value means that 10% by volume of the particles of the measured sample have a particle diameter below the value reported as D[v, 0.1]. Accordingly, the D[v, 0.5] value means that 50% by volume of the particles of the measured sample have a particle diameter below the value reported as D[v, 0.5], and the D[v, 0.9] value means that 90% by volume of the particles of the measured sample have a particle diameter below the value reported as D[v, 0.9]. The D[4,3] value is the volume-weighted average determined by means of SLS, which is also referred to as the De Brouckere mean and corresponds to the mass average for the particles of the invention. The D[3, 2] value is the surface-weighted average determined by means of SLS, which is also referred to as the Sauter diameter.

Process step i.

In the first step i. of the process of the present invention at least one aroma chemical and an organic polymer composition, which contains at least one polyester as a main constituent are dissolved in a volatile organic solvent. Thereby, a solution of the aroma chemical and the organic polymer composition in the organic solvent is obtained.

The volatile organic solvent used for dissolving the polymer composition and the aroma chemical has a boiling point lower than the boiling point of the aroma chemical, in particular a boiling point which is at least 10° C. more particularly at least 15° C. or at least 20° C., especially at least 30° C. lower than the boiling point of the aroma chemical at 1013 mbar. Typically, the boiling point of the volatile organic solvent is lower than 100° C. at 1013 mbar. Moreover, the organic solvent has a solubility in water of at most or less than 100 g/L, e.g. 1 mg/L to 100 g/L at 20° C. and 1013 mbar, in particular 1 mg/L to 80 g/L at 20° C. and 1013 mbar.

In addition, the water-immiscible solvent preferably has a boiling point of at least 30° C. at 1013 mbar. In particular, the organic solvent has a boiling point at 1013 mbar in the range of 35 to 85° C., in particular in the range of 38 to 80° C.

According to the general knowledge of those skilled in the art, solvents are chemically inert to the substances to be dissolved therein; that is to say, they merely serve for dilution or dissolution. Free radically-polymerizable monomers are not solvents in the context of the invention.

Preference is given to aprotic non-polar and aprotic polar solvents or solvent mixtures having the above solubility and boiling points. Preferred solvents are, for example, dichloromethane, chloroform (trichloromethane), ethyl acetate, pentane, n-hexane, n-heptane, cyclohexane, methyl tert-butyl ether, diethyl ether, diisopropyl ether and benzene, or mixtures of two or more of these solvents with one another. Preferably, the solvent comprises dichloromethane as a main constituent. Especially, the solvent is dichloromethane. Further suitable solvent mixtures are those that form an azeotrope having a boiling point within the range of 35 to 85° C., in particular in the range of 38 to 80° C. at 1013 mbar and whose average solubility in water, i.e. the weight average of the water solubilities of the solvents in the mixture at 20° C. and 1013 mbar, is at most 100 g/L. The aforementioned solvents may be used in combination with minor amounts of an organic solvent having a solubility in water of 100 g/L or higher, e.g. up to 300 g/L at 20° C. and 1013 mbar, as long as its amount is less than 50% by weight of the total amount of the organic solvent, and the boiling point of the combination is in the above ranges. Such a mixture may be a mixture of one of the aformentioned hydrocarbon solvents and methyl ethyl ketone (MEK), preferably in the weight ratio of hydrocarbon solvent to MEK in the range of 1.1:1 to 4:1.

The organic polymer composition may in principle be any organic polymer or a mixture of organic polymers, as long as the composition is soluble in the organic solvent or solvent mixture. Preferably, the organic polymer composition, hereinafter also termed “wall material”, has a solubility in the respective volatile organic solvent or solvent mixture of at least 50 g/L, in particular at least 100 g/L at 25° C. and 1013 mbar and may be completely miscible with the volatile organic solvent or solvent mixture. In particular, the organic polymer composition has a solubility in dichloromethane of at least 50 g/L, in particular at least 100 g/L at 25° C. and 1013 mbar.

In general, the polymer composition is dissolved in the volatile organic solvent or solvent mixture such that its concentration is in the range of 1 to 250 g/kg, in particular in the range of 2 to 150 g/kg, especially 3 to 100 g/kg, based on the total weight of the solution.

Preferably, the organic polymer composition essentially consists of one or more thermoplastic organic polymers but it may also contain small amounts of non-thermoplastic materials, e.g. duroplastic polymers, as long as these components do not impart the thermoplasticity of the organic polymeric composition. In particular, the amount of non-thermoplastic material will not exceed 10% by weight, in particular 5% by weight of the total amount of thermoplastic organic polymeric material which forms the microparticles. The term “thermoplastic” is well understood by a person of skill in the art that a thermoplastic polymer can be melted and recast almost indefinitely. Thermoplastic polymers soften or melt when heated and harden upon cooling and can be formed or molded in the softened or molten state.

According to the invention, the polymer composition comprises one polyester as a main constituent. In particular, the amount of polyester in the polymer composition is at least 60% by weight, in particular at least 70% by weight, especially at least 80% by weight or at least 90% by weight, and may be up to 100% by weight, based on the total weight of the polymer composition used in the process of the present invention.

Polyesters suitable for the purpose of the present invention include aliphatic polyesters, semiaromatic polyesters and aromatic polyesters. The term polyester also includes polyesteramides, polyetheresters, polyesterurethanes and polyester carbonates, including aliphatic and semiaromatic polyesteramides, aliphatic and semiaromatic polyetheresters, aliphatic and semiaromatic polyesterurethanes and aliphatic and semiaromatic aliphatic polyester carbonates. Further polymers which may be present in the polymer composition include in particular condensation polymers different from polyesters, including polyamides, polycarbonates, but also addition polymers, such as polystyrenes, polyacrylates, polyolefins, polyureas and polyurethanes, modified polysaccharides and proteins and blends of the aforementioned polymers.

Preferably, the polymer composition is biodegradable or contains a biodegradable polyester as the main constituent. The term “biodegradable” is understood to mean that the substance in question, the filled microparticles according to the invention, in the test of OECD Guideline 301B from 1992 (measurement of evolution of CO₂ on composting in a mineral slurry and comparison with the theoretical maximum possible evolution of CO₂) after 28 days and 25° C. undergoes biodegradation of at least 5%, particularly at least 10% and especially at least 20%.

Preferably, the polymer composition comprises at least one polyester as a main constituent, which has a glass transition temperature or melting temperature in the range from 45 to 160° C., in particular in the range from 50 to 150° C. If the polymer has a melting point, i.e. is semicrystalline or crystalline, it preferably has a melting or crystallization temperature in the range from 45 to 160° C., in particular in the range from 60 to 150° C. If the polymer is amorphous, it preferably has a glass transition temperature in the range from 45 to 160° C., in particular in the range from 50 to 150° C. The glass transition temperature here is typically determined by means of dynamic differential calorimetry (DSC) to DIN EN ISO 11357-1:2017-02. The melting temperature here is typically determined by means of dynamic differential calorimetry (DSC) to DIN EN ISO 11357-3:2018-07.

In one embodiment, the polymer compositions comprise at least one aliphatic polyester, especially at least one aliphatic-aliphatic polyester, which is in particular the main constituent of the polymer composition. Aliphatic-aliphatic polyesters are understood to mean polyesters based on aliphatic dicarboxylic acids and aliphatic dihydroxyl compounds, and polyesters based on mixtures of aliphatic dicarboxylic acids with aliphatic dicarboxylic acids and aliphatic dihydroxyl compounds. These polymers may be present individually or in the mixtures thereof. Wall materials based on aliphatic polyesters of this kind are the most readily biodegradable systematic polymers, and hence so are the filled microparticles produced therefrom.

As used herein, the terms “aliphatic-aliphatic polyester”, “aliphatic copolyester” and “aliphatic-aliphatic copolyester” are used synonymously.

Examples of aliphatic carboxylic acids and the ester-forming derivatives, which are suitable for the preparation of aliphatic-aliphatic polyesters, are those having 2 to 18 carbon atoms, preferably 4 to 10 carbon atoms. They may be either linear or branched. However, it is also possible in principle to employ dicarboxylic acids having a greater number of carbon atoms, for example having up to 30 carbon atoms.

Preference is given to aliphatic-aliphatic polyesters in which the aliphatic dicarboxylic acid is selected from succinic acid, adipic acid, azelaic acid, sebacic acid, brassylic acid and mixtures thereof. Particular preference is given to succinic acid, adipic acid and sebacic acid and mixtures thereof. To prepare the aliphatic-aliphatic polyesters, instead of the dicarboxylic acids, their respective ester-forming derivatives or mixtures thereof with the dicarboxylic acids may also be used.

Examples of aliphatic diols which are suitable for the preparation of the aliphatic-aliphatic polyesters are, for example, branched or linear alkanediols having 2 to 12 carbon atoms, preferably 4 to 6 carbon atoms, or cycloalkanediols having 5 to 10 carbon atoms. Examples of suitable alkanediols are ethylene glycol, 1,2-propanediol, 1,3-propanediol, 1,2-butanediol, 1,4-butanediol, 1,5-pentanediol, 2,4-dimethyl-2-ethylhexane-1,3-diol, 2,2-dimethyl-1,3-propanediol, 2-ethyl-2-butyl-1,3-propanediol, 2-ethyl-2-isobutyl-1,3-propanediol, 2,2,4-trimethyl-1,6-hexanediol, especially ethylene glycol, 1,3-propanediol, 1,4-butanediol and 2,2-dimethyl-1,3-propanediol (neopentyl glycol). Examples of cycloalkanediols are cyclopentanediol, 1,4-cyclohexanediol, 1,2-cyclohexanedimethanol, 1,3-cyclohexanedimethanol, 1,4-cyclohexanedimethanol and 2,2,4,4-tetramethyl-1,3-cyclobutanediol. The aliphatic-aliphatic polyesters may also comprise mixtures of different alkanediols condensed. In particular, preference is given to 1,4-butanediol, especially in combination with one or two aliphatic dicarboxylic acids selected from succinic acid, adipic acid and sebacic acid, as component a1).

Examples of particularly preferred aliphatic-aliphatic polyesters are polybutylene succinate adipate, polybutylene succinate, polybutylene sebacate and polybutylene succinate sebacate.

The preferred aliphatic polyesters, in particular the aliphatic copolyesters, often have a number average molecular weight Mn in the range from 1000 to 100 000 g/mol, particularly in the range from 1000 to 75 000 g/mol, especially in the range from 1500 to 6500 g/mol.

The values given for the number average molecular weight Mn and for the weight average molecular weight Mw of polyesters refer to the molecular weights of the respective polyesters as determined by GPC using tetrahydrofurane as an eluent and polystyrene or polymethylmethycrylate of defined molecular weight as standards, if not stated otherwise.

The preferred aliphatic polyesters, in particular the aliphatic copolyesters, often have a melting point in the range of 50 to 120° C., particularly in the range of 55 to 115° C., as determined by DSC.

Particularly preferred aliphatic polyesters include in particular aliphatic copolyesters, e.g. polyester polyols that are partially or highly crystalline and solid. Such aliphatic copolyesters have hydroxyl number in the range of 10 to 34, particularly in the range of 27 to 34 mg KOH/g, as determined according to DIN EN ISO 4629-2.

Acid number of suitable aliphatic polyesters, in particular of suitable aliphatic copolyesters is at most 3, particularly at most 2 mg KOH/g, as determined according to DIN EN ISO 2114.

Melting point of suitable aliphatic polyesters, in particular of suitable aliphatic copolyesters is in the range of 50 to 120° C., particularly in the range of 55 to 115° C., as determined by DSC.

Viscosity of suitable aliphatic polyesters, in particular of suitable aliphatic copolyesters is at 130° C. in the range of 0.2 to 5 Pa-s, particularly 0.3 to 4 Pa-s, or at 80° C. in the range of 1 to 16 Pa-s, particularly in the range of 2 to 15 Pa-s, as determined by parallel plate.

Suitable aliphatic polyesters, in particular aliphatic copolyesters have a molecular weight Mn in the range of 1000 to 9000 g/mol, particularly in the range of 1500 to 6500 g/mol, as determined from hydroxyl number. Preferably, the molecular weight of suitable aliphatic copolyester is 3500, as determined from hydroxyl number.

The suitable aliphatic polyesters are solid, high crystalline aliphatic copolyesters which are commercially available e.g. as Dynacoll® 7300 grades of Evonik, such as DYNACOLL® 7320, DYNACOLL® 7330, DYNACOLL® 7340, DYNACOLL® 7360, DYNACOLL® 7361, DYNACOLL® 7363, DYNACOLL® 7365, DYNACOLL® 7380, DYNACOLL® 7381, DYNACOLL® 7390, DYNACOLL® 7391.

Preferred polymer compositions comprise at least one semiaromatic polyester, which is in particular the main constituent of the polymer composition. Semiaromatic polyesters are also referred to as aliphatic-aromatic polyesters, i.e. polyesters based on aromatic dicarboxylic acids and aliphatic dihydroxyl compounds, and polyesters based on mixtures of aromatic dicarboxylic acids with aliphatic dicarboxylic acids and aliphatic dihydroxyl compounds. Aliphatic-aromatic polyesters are preferably polyesters based on mixtures of aliphatic dicarboxylic acids with aromatic dicarboxylic acids and aliphatic dihydroxyl compound. These polymers may be present individually or in the mixtures thereof. Wall materials based on semiaromatic polyesters of this kind are typically biodegradable for the purposes of this invention, and hence so are the filled microparticles produced therefrom.

Preferably, “semi-aromatic polyesters” shall also be understood to mean polyester derivatives such as polyetheresters, polyesteramides or polyetheresteramides and polyesterurethanes, as described, for example, in WO 2012/2013506. The suitable semi-aromatic polyesters include linear, non-chain-extended polyesters, as described for example in WO 92/09654. Preference is given to chain-extended and/or branched aliphatic-aromatic polyesters. The latter are known from WO 96/15173, WO 96/15174, WO 96/15175, WO 96/15176, WO 96/21689, WO 96/21690, WO 96/21691, WO 96/21692, WO 96/25446, WO 96/25448 and WO 98/12242, to which explicit reference is made. Likewise considered are mixtures of different aliphatic-aromatic polyesters. Interesting recent developments are based on renewable raw materials and are described inter alia in WO 2006/097353, WO 2006/097354 and WO 2010/034710.

Particularly preferred semi-aromatic polyesters include polyesters comprising as essential components:

A) an acid component formed from
a1) 30 to 99 mol % of at least one aliphatic dicarboxylic acid or the ester-forming derivatives thereof or mixtures thereof,
a2) 1 to 70 mol % of at least one aromatic dicarboxylic acid or the ester-forming derivative thereof or mixtures thereof and

B) at least one diol component selected from C₂- to C₁₂-alkanediols and

C) optionally a component selected from
c1) a compound having at least three groups capable of ester formation,
c2) a diisocyanate or polyisocyanate,
c3) a diepoxide or polyepoxide.

Aliphatic dicarboxylic acids and the ester-forming derivatives thereof (a1) that are generally considered are those having 2 to 18 carbon atoms, preferably 4 to 10 carbon atoms. They may be either linear or branched. However, it is also possible in principle to employ dicarboxylic acids having a greater number of carbon atoms, for example having up to 30 carbon atoms.

Examples of aliphatic dicarboxylic acids and the ester-forming derivatives include: oxalic acid, malonic acid, succinic acid, 2-methylsuccinic acid, glutaric acid, 2-methylglutaric acid, 3-methylglutaric acid, α-ketoglutaric acid, adipic acid, pimelic acid, azelaic acid, sebacic acid, brassylic acid, fumaric acid, 2,2-dimethylglutaric acid, suberic acid, diglycolic acid, oxaloacetic acid, glutamic acid, aspartic acid, itaconic acid and maleic acid, their anhydrides and their C₁-C₄-alkyl esters. These dicarboxylic acids or the ester-forming derivatives thereof may be used individually or as a mixture of two or more thereof.

It is preferable to employ succinic acid, adipic acid, azelaic acid, sebacic acid, brassylic acid or their respective ester-forming derivatives or mixtures thereof. It is particularly preferable to employ succinic acid, adipic acid or sebacic acid or the respective ester-forming derivatives thereof or mixtures thereof. Succinic acid, azelaic acid, sebacic acid and brassylic acid additionally have the advantage that they are obtainable from renewable raw materials.

The aromatic dicarboxylic acids or the ester-forming derivatives thereof (a2) may be used individually or as a mixture of two or more thereof. Particular preference is given to using terephthalic acid or the ester-forming derivatives thereof such as dimethyl terephthalate.

Generally, the diols (B) are selected from branched or linear alkanediols having 2 to 12 carbon atoms, preferably 4 to 6 carbon atoms, or cycloalkanediols having 5 to 10 carbon atoms. Examples of suitable alkanediols are ethylene glycol, propane-1,2-diol, propane-1,3-diol, butane-1,2-diol, butane-1,4-diol, pentane-1,5-diol, 2,4-dimethyl-2-ethylhexane-1,3-diol, 2,2-dimethylpropane-1,3-diol, 2-ethyl-2-butylpropane-1,3-diol, 2-ethyl-2-isobutylpropane-1,3-diol, 2,2,4-trimethylhexane-1,6-diol, especially ethylene glycol, propane-1,3-diol, butane-1,4-diol and 2,2-dimethylpropane-1,3-diol (neopentyl glycol). Examples of suitable cylcoalkanediols are cyclopentanediol, cyclohexane-1,4-diol, cyclohexane-1,2-dimethanol, cyclohexane-1,3-dimethanol, cyclohexane-1,4-dimethanol and 2,2,4,4-tetramethylcyclobutane-1,3-diol. The aliphatic-aromatic polyesters may also include combinations of different alkanediols or cycloalkanediols. Particular preference is given to butane-1,4-diol, especially in combination with adipic acid as component a1), and butane-1,4-diol or propane-1,3-diol, especially in combination with sebacic acid as component a1). Propane-1,3-diol also has the advantage that it is obtainable as a renewable raw material.

More preferably, the aliphatic-aromatic polyester is selected from polybutylene azelateco-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST). The aforementioned aliphatic-aromatic polyesters have VST/A50 values in the range of 50 to 160° C., in particular in the range of 55 to 150° C.

The preferred aliphatic-aromatic polyesters are characterized by a number average molecular weight Mn in the range from 1000 to 100 000 g/mol, especially in the range from 1000 to 75 000 g/mol, preferably in the range from 1500 to 50 000 g/mol.

Preferably, at least one of the polymers of the polymer composition has a glass transition temperature or a melting point in the range from 45 to 160° C., in particular in the range from 50 to 150° C.

In a preferred group of embodiments, the polymer composition essentially consists of an aliphatic-aromatic polyester. In this group of embodiments the wall material of the microparticles consists in particular to an extent of at least 95% by weight, especially to an extent of at least 99% by weight, based on the wall material of an aliphatic-aromatic polyester. Preferably, the aliphatic-aromatic polyester has a glass transition temperature or a melting point in the range from 45 to 160° C., in particular in the range from 50 to 150° C.

In a more preferred group of embodiments, the polymer composition comprises or essentially consists of a combination of an aliphatic-aromatic polyester and at least one further thermoplastic polymer which is different from aliphatic-aromatic polyesters. In this group of embodiments the polymer composition essentially consists of, in particular to an extent of at least 95% by weight, especially to an extent of at least 99% by weight, based on the polymer composition, of the combination of at least one aliphatic-aromatic polyester and at least one further polymer. Preferably, the amount of aliphatic-aromatic polyester in the mixture amounts to at least 30% of the combination, e.g. from 50 to 99% of the combination. In particular, the mass ratio of the aliphatic-aromatic polyester to the at least one further polymer which is different from aliphatic-aromatic polyesters is in the range from 30:70 to 99:1 or in the range from 30:70 to 80:20, in particular in the range from 35:65 to 75:25 and especially in the range form 40:60 to 70:30.

The further polymer is usually a thermoplastic polymer. Preferably, the further polymer, which is not an aliphatic-aromatic polyester, has a glass transition temperature or a melting point in the range from 45 to 160° C., in particular in the range from 50 to 150° C.

Examples of said further polymer that is not an aliphatic-aromatic polyester and which is thermoplastic include but are not limited to: polyacrylates, polyamides, polycarbonates, polystyrenes, aliphatic polyesters, in particular aliphatic-aliphatic copolyesters, aliphatic polyetheresters, aliphatic polyesteramides, aliphatic polylactones, aliphatic polylactones, polyanhydrides, aromatic/aromatic polyesters, polyolefines, polyureas, polyurethanes, modified polysaccharides and proteins.

The further polymer is preferably selected from the group consisting of aliphatic polyesters, aliphatic polyanhydrides, aliphatic polyetheresters, aliphatic polyesteramides, modified polysaccharides and proteins and mixtures thereof. In particular, the further polymer is selected from the group consisting of polyesters of hydroxycarboxylic acids, aliphatic-aliphatic polyesters, polylactones, poly(p-dioxanones), polyanhydrides and aliphatic polyesteramides. The at least one further polymer is especially selected from the group consisting of aliphatic polyesters, especially polylactic acid, polyhydroxy fatty acids, aliphatic-aliphatic polyesters, polyhydroxy acetic acids and poly-C₆-C₁₂-lactones and mixtures thereof.

In a particular group of embodiments, the further polymer comprises at least one aliphatic polyester, which is selected from the group consisting of polyhydroxy acetic acids and polylactic acid and copolymers thereof. Amongst these, polylactic acids, also termed polylactides (also termed PLA) and copolymers of lactic acid (PLA copolymers), in particular copolymers of lactic acid and glycolic acid, i.e. polylactid-co-glycolid (also termed PLGA). Amongst PLA and PLA-copolymers PLA is preferred. Polylactic acid having a molecular weight of 30 000 to 120 000 daltons and a glass transition temperature (Tg) in the range from 50 to 65° C. is particularly suitable. Most preferably, amorphous polylactic acid having a proportion of D-lactic acid greater than 9% is used.

Preferred components in the mixtures with the at least one aliphatic-aromatic polyester are polyhydroxyacetic acid, PLA copolymers (polylactide and polylactic acid copolymers) and PLGA copolymers, and here especially polylactide copolymers. Polylactic acid having a molecular weight of 30 000 to 120 000 daltons and a glass transition temperature (Tg) in the range from 50 to 65° C. is particularly suitable. Most preferably, amorphous polylactic acid having a proportion of D-lactic acid greater than 9% is used. Polyhydroxyalkanoates are understood to mean primarily poly-4-hydroxybutyrates and poly-3-hydroxybutyrates, and also include copolyesters of the aforementioned hydroxybutyrates with 3-hydroxyvalerates (P(3HB)-co-P(3HV)) or 3-hydroxyhexanoate.

According to a further preferred group of embodiments, the further polymer comprises an aliphatic polyester of the group of polyhydroxy fatty acids. Polyhydroxy fatty acids are polyesters based on hydroxy fatty acids, which have from 1 to 18, in particular from 1 to 6 carbon atoms between the carbon atom bearing the OH group and the carbon atom of the carboxyl group. Polyhydroxy fatty acids also include polyesters of 2-hydroxybutyric acid, in particular their homopolymers. Accordingly, polylactic acid and polyhydroxyacetic acid are not polyhydroxy fatty acids. Typically, polyhydroxy fatty acid comprise repeating monomer units of the formula (1b)

[—O—CHR—(CH₂)ₘ—CO—] (1a)

[—O—CHR′—CO—] (1b)

where R is hydrogen or a linear or branched alkyl group having 1 to 20, preferably 1 to 16 carbon atoms, especially 1 to 6 carbon atoms, R′ is a linear or branched alkyl group having 2 to 20, preferably 2 to 16 carbon atoms, preferably 2 to 6 carbon atoms and m=numbers from 1 to 18, preferably 1, 2, 3, 4, 5 and 6.

Polyhydroxy fatty acid include homopolymers (also termed homopolyesters), i.e. polyhydroxy fatty acids of identical types of hydroxy fatty acid monomers and copolyesters, i.e. polyhydroxy fatty acids of different types of hydroxy fatty acid monomers. Polyhydroxy fatty acids can be used in arbitrary mixtures of polyhydroxy fatty acids.

Polyhydroxy fatty acid usually have a weight average molecular weight M, in the range of 5000 to 1 000 000, in particular in the range of 30 000 to 1 000 000, or in the range of 70 000 to 1 000 000, preferably in the range of 100 000 to 1000 000 or in the range of 300 000 to 600 000 and/or having melting temperatures in the range of 100 to 180° C.

In one embodiment of the invention, the at least one polyhydroxy fatty acid is selected from the group consisting of
poly-3-hydroxypropionates (P3HP);
polyhydroxybutyrates (PHB);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid and at least one monomer selected from the group consisting of 3-hydroxypropionic acid, hydroxyvaleric acid, hydroxyhexanoic acid, hydroxyoctanoic acid and hydroxyoctadecaniuc acid;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acid;
Poly-C₆-C₁₂-lactones, especially polycaprolactone.

Suitable polyhydroxybutyrates (PHB) may be selected from the group consisting of poly(2-hydroxybutyrates) (P2HB), poly(3-hydroxybutyrates) (P3HB), poly(4-hydroxybutyrates) (P4HB) and copolymers of at least 2 hydroxybutyric acids selected from the group consisting of 2-hydroxybutyric acid, 3-hydroxybutyric acid and 4-hydroxybutyric acid. Further suitable are copolymers of 3-hydroxybutyric acid and 4-hydroxybutyric acid. These copolymers are characterized by the following abbreviations: [P(3HB-co-4HB)], where 3HB is 3-hydroxybutyrate and 4HB is 4-hydroxybutyrates.

Poly(3-hydroxybutyrates) are marketed for example by PH B Industrial under the brand Biocycle® and by Tianan under the name Enmat®. Poly-3-hydroxybutyrate-co-4-hydroxybutyrates are known from Metabolix in particular. They are sold under the trade name Mirel®.

Suitable polyhydroxyvalerates (PHV) may be selected from the group consisting of homopolymers of 3-hydroxyvaleric acid [=poly(3-hydroxyvalerates) (P3HV)], homopolymers of 4-hydroxyvaleric acid [=poly(4-hydroxyvalerates) (P4HV)]; homopolymers of 5-hydroxyvaleric acid [=poly(5-hydroxyvalerates) (P5HV)]; homopolymers of 3-hydroxymethylvaleric acid [=poly(3-hydroxymethylvalerates) (P3MHV)]; copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid.

Suitable polyhydroxyhexanoates (PHHx) may be selected from the group consisting of poly(3-hydroxyhexanoates) (P3HHx), poly(4-hydroxyhexanoates) (P4HHx), poly(6-hydroxyhexanoates) (P6HHx) and copolymers of at least 2 hydroxyhexanoic acids selected from the group consisting of 3-hydroxyhexanoic acid, 4-hydroxyhexanoic acid and 6-hydroxyhexanoic acid.

Suitable polyhydroxyoctanoates (PHO) may be selected from the group consisting of poly(3-hydroxyoctanoates) (P3HO), poly(4-hydroxyoctanoates) (P4HO), poly(6-hydroxyoctanoates) (P6HO) and copolymers of at least 2 hydroxyoctanoic acids selected from the group consisting of 3-hydroxyoctanoic acid, 4-hydroxyoctanoic acid and 6-hydroxyoctanoic acid.

Suitable copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids may be selected from the group consisting of
copolyesters of 4-hydroxybutyric acid with 3-hydroxyvaleric acid [P(4HB-co-3HV)]
copolyesters of 3-hydroxybutyric acid with 3-hydroxyvaleric acid [P(3HB-co-3HV)]
copolyesters of 4-hydroxybutyric acid with 3-hydroxyhexanoic acid [P(4HB-co-3HHx)]
copolyesters of 3-hydroxybutyric acid with 3-hydroxyhexanoic acid [P(3HB-co-3HHx)]
copolyesters of 4-hydroxybutyric acid with 3-hydroxyoctanoic acid [P(4HB-co-3HO)] and
copolyesters of 3-hydroxybutyric acid with 3-hydroxyoctanoic acid [P(3HB-co-3HO)]
copolyesters of 4-hydroxybutyric acid with 3-hydroxyoctadecanoic acid [P(4HB-co-3HOD)] and
copolyesters of 3-hydroxybutyric acid with 3-hydroxyoctadecanoic acid [P(3HB-co-3HOD)]

Preference is given to using poly-3-hydroxybutyrate-co-3-hydroxyhexanoate having a 3-hydroxyhexanoate proportion of 1 to 20 and preferably of 3 to 15 mol % based on the total amount of polyhydroxy fatty acid. Such poly-3-hydroxybutyrate-co-3-hydroxyhexanoates [P(3HB-co-3HHx] are known from Kaneka and are commercially available under the trade names Aonilex™ X131A and Aonilex™ X151A.

Suitable copolyesters of hydroxyvaleric acid are preferably copolyesters of 4-hydroxyvaleric acid and/or 3-hydroxyvaleric acid with at least one monomer selected from the group consisting of 3-hydroxypropionic acid, hydroxyhexanoic acids, hydroxyoctanoic acids, especially 3-hydroxyoctanoic acid and hydroxyoctadecanoic acids.

Suitable copolyesters of hydroxyhexanoic acid are preferably copolyesters of 3-hydroxyhexanoic acid with at least one monomer selected from the group consisting of 3-hydroxypropionic acid and hydroxyoctanoic acid, preferably 3-hydroxyoctanoic acid and hydroxyoctadecanoic acids.

According to a particular preferred group of embodiments, the further polymer comprises an aliphatic polyester of the group of polylactones, in particular from the group of poly-C₆-C₁₂-lactones, especially polycaprolactones (PCL). Polylactones refer to polyesters obtainable by ring-opening polymerization of lactones, in particular C₆-C₁₂-lactones, especially epsilon-caprolactone (ε-caprolactone). Polycaprolactones are therefore polyhydroxy fatty acids with repeating monomer units of the general formula (1) [—O—CHR—(CH₂)ₘ—CO—], in which m is 4 to 10, in case of caprolactone m=4, and R is hydrogen. In the context of the invention, the term polycaprolactone is understood to mean both homopolymers of epsilon-caprolactone and copolymers of epsilon-caprolactone. Suitable copolymers are, for example, copolymers of epsilon-caprolactone with monomers selected from the group consisting of lactic acid, lactide, hydroxyacetic acid and glycolide. Polycaprolactones are marketed, for example, by Perstorp under the brand name Capa™ or by Daicel under the brand name Celgreen™.

In a preferred embodiment, the at least one polyhydroxy fatty acid is a polycaprolactone.

In one group of embodiments of the invention, the at least one polyhydroxy fatty acid is selected from the group consisting of
poly(3-hydroxypropionates) (P3HP);
polyhydroxybutyrates (PHB), including poly(4-hydroxybutyrates) and poly(3-hydroxybutyrates);
polyhydroxyvalerates (PHV);
polyhydroxyhexanoates (PHHx);
polyhydroxyoctanoates (PHO);
polyhydroxyoctadecanoates (PHOD);
copolyesters of hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids;
copolyesters of hydroxyvaleric acid with at least one monomer selected from the group consisting of 3-hydroxypropionic acid, hydroxyhexanoic acids, hydroxyoctanoic acids and hydroxyoctadecanoic acids;
copolyesters of hydroxyhexanoic acid with at least one monomer selected from the group consisting of 3-hydroxypropionic acid, hydroxyoctanoic acid and hydroxyoctadecanoic acid; and
polycaprolactones.

In one embodiment of the invention, the at least one polyhydroxy fatty acid is selected from the group consisting of poly(3-hydroxypropionates) (P3HP); poly(2-hydroxybutyrates) (P2HB); copolymers of at least 2 hydroxybutyric acids selected from the group consisting of 2-hydroxybutyric acid, 3-hydroxybutyric acid and 4-hydroxybutyric acid; copolymers of 3-hydroxybutyric acid and 4-hydroxybutyric acid; poly(3-hydroxyvalerates) (P3HV); poly(4-hydroxyvalerates) (P4HV); poly(5-hydroxyvalerates) (PSHV); poly(3-hydroxymethylvalerates) (P3MHV); copolymers of at least 2 hydroxyvaleric acids selected from the group consisting of 3-hydroxyvaleric acid, 4-hydroxyvaleric acid, 5-hydroxyvaleric acid and 3-hydroxymethylvaleric acid; poly(3-hydroxyhexanoates) (P3HHx); poly(4-hydroxyhexanoates) (P4HHx); poly(6-hydroxyhexanoates) (P6HHx); copolymers of at least 2 hydroxyhexanoic acids selected from the group consisting of 3-hydroxyhexanoic acid, 4-hydroxyhexanoic acid and 6-hydroxyhexanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; poly(3-hydroxyoctanoates) (P3HO); poly(4-hydroxyoctanoates) (P4HO); poly(6-hydroxyoctanoates) (P6HO); copolymers of at least 2 hydroxyoctanoic acids selected from the group consisting of 3-hydroxyoctanoic acid, 4-hydroxyoctanoic acid and 6-hydroxyoctanoic acid; copolyesters of 2-hydroxybutyric acid with at least one monomer selected from the group consisting of 3-hydroxypropionic acid, hydroxyvaleric acids, hydroxyhexanoic acids, hydroxyoctanoic acids and hydroxyoctadecanoic acids; copolyesters of 4-hydroxybutyric acid with 3-hydroxyoctanoic acid [P(4HB-co-3HO)], copolyesters of 3-hydroxybutyric acid with 3-hydroxyoctanoic acid [P(3HB-co-3HO)], copolyesters of 4-hydroxybutyric acid with 3-hydroxyoctadecanoic acid [P(4HB-co-3HOD)], copolyesters of 3-hydroxybutyric acid with 3-hydroxyoctadecanoic acid [P(3HB-co-3HOD)]; copolyesters of hydroxyvaleric acid, especially of 3-hydroxyvaleric acid or 4-hydroxyvaleric acid with at least one monomer selected from the group consisting of 3-hydroxypropionic acid, hydroxyhexanoic acids, hydroxyoctanoic acids and hydroxyoctadecanoic acids; copolyesters of 3-hydroxyhexanoic acid with at least one monomer selected from the group consisting of 3-hydroxypropionic acid, hydroxyoctanoic acid, preferably 3-hydroxyoctanoic acid and hydroxyoctadecanoic acids; and polycaprolactones.

According to a further group of embodiments, the further polymer of the polymer composition comprises at least one polyhydroxy fatty acid, which is selected from the group consisting of polyhydroxy alkanoates. Polyhydroxy alkanoates mainly refer to poly-4-hydroxybutyrates and poly-3-hydroxybutyrates and copolyesters of the aforementioned hydroxybutyrates with 3-hydroxyvalerates (P(3HB)-co-P(3HV)) or 3-hydroxyhexanoates. Usually, polyhydroxy alkanoates have a weight average molecular weight Mw in the range of 30 000 to 1 000 000 g/mol and in particular in the range of 100 000 to 600 000 g/mol.

According to a further group of embodiments, the further polymer of the polymer composition comprises at least one aliphatic-aliphatic polyester. Aliphatic-aliphatic polyesters are understood to mean polyesters based on aliphatic dicarboxylic acids and aliphatic dihydroxyl compounds, and polyesters based on mixtures of aliphatic dicarboxylic acids with aliphatic dicarboxylic acids and aliphatic dihydroxyl compounds.

Examples of aliphatic carboxylic acids which are suitable for the preparation of aliphatic-aliphatic polyesters are the aliphatic dicarboxylic acids mentioned under (a1), especially those having 2 to 18 carbon atoms, preferably 4 to 10 carbon atoms. Preference is given to aliphatic-aliphatic polyesters in which the aliphatic dicarboxylic acid is selected from succinic acid, adipic acid, azelaic acid, sebacic acid, brassylic acid and mixtures thereof. Particular preference is given to succinic acid, adipic acid and sebacic acid and mixtures thereof. To prepare the aliphatic-aliphatic polyesters, instead of the dicarboxylic acids, their respective ester-forming derivatives or mixtures thereof with the dicarboxylic acids may also be used.

Examples of aliphatic diols which are suitable for the preparation of the aliphatic-aliphatic polyesters are the diols mentioned as component (B), for example branched or linear alkanediols having 2 to 12 carbon atoms, preferably 4 to 6 carbon atoms, or cycloalkanediols having 5 to 10 carbon atoms. Examples of suitable alkanediols are ethylene glycol, 1,2-propanediol, 1,3-propanediol, 1,2-butanediol, 1,4-butanediol, 1,5-pentanediol, 2,4-dimethyl-2-ethylhexane-1,3-diol, 2,2-dimethyl-1,3-propanediol, 2-ethyl-2-butyl-1,3-propanediol, 2-ethyl-2-isobutyl-1,3-propanediol, 2,2,4-trimethyl-1,6-hexanediol, especially ethylene glycol, 1,3-propanediol, 1,4-butanediol and 2,2-dimethyl-1,3-propanediol (neopentyl glycol). Examples of cycloalkanediols are cyclopentanediol, 1,4-cyclohexanediol, 1,2-cyclohexanedimethanol, 1,3-cyclohexanedimethanol, 1,4-cyclohexanedimethanol and 2,2,4,4-tetramethyl-1,3-cyclobutanediol. The aliphatic-aliphatic polyesters may also comprise mixtures of different alkanediols condensed in. Particular preference is given to 1,4-butanediol, especially in combination with one or two aliphatic dicarboxylic acids selected from succinic acid, adipic acid and sebacic acid, as component a1).

Examples of particularly preferred aliphatic-aliphatic polyesters are polybutylene succinate adipate, polybutylene succinate, polybutylene sebacate and polybutylene succinate sebacate.

The preferred aliphatic-aliphatic polyesters often have a molecular weight Mn in the range from 1000 to 100 000 g/mol, particularly in the range from 1000 to 75 000 g/mol, especially in the range from 1500 to 50 000 g/mol.

According to a further group of embodiments the further polymer of the polymer composition comprises at least one poly-p-dioxanone (poly-1,4-dioxan-2-one). Poly-p-dioxanones (poly-1,4-dioxan-2-one) refer to poly(ether-esters) obtainable by ring-opening polymerization of 1,4-dioxan-2-one. In the context of the present invention, the term poly(p-dioxanones) are understood to mean homopolymers of 1,4-dioxan-2-one, which have the general structural unit [—O—CH₂—CH₂—O—CH₂—CO-]ₙ. In the context of the present invention, the term poly(p-dioxanones) is also understood to mean copolymers of 1,4-dioxan-2-one with lactone monomers. Particularly suitable are copolymers of 1,4-dioxan-2-one with at least one further monomer selected from the group consisting of glycolide, lactide and epsilon-caprolactone.

According to a further group of embodiments, the further polymer of the polymer composition comprises at least one polyanhydride. Polyanhydrides refer to polymers having the general structural unit

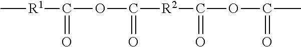

as characteristic base units of the main chain. R¹ and R² can be the same or different aliphatic or aromatic radicals. Suitable polyanhydrides are described in Kumar et al, Adv. Drug Delivery Reviews 54 (2002), pp. 889-910. Particularly suitable are the polyanhydrides described in Kumar et al. Adv. Drug Delivery Reviews 54 (2002), on p. 897, which is fully incorporated here by way of reference. In one embodiment of the invention, the polyanhydride is selected from the group of aliphatic polyanhydrides, especially from the group consisting of polysebacic acid and polyadipic acid.

A further group of polymers, which can be used in combination with the aliphatic-aromatic polyester in the polymer composition, are polyester amides, in particular aliphatic polyester amides. Aliphatic polyesteramides are copolymers bearing both amide and ester functions. Suitable aliphatic polyesteramides are particularly polyesteramides obtained by polycondensation of ε-caprolactam, adipic acid, 1,4-butanediol and hexamethylene diamine, and polyesteramides obtained by polycondensation of adipic acid, 1,4-butanediol, diethylene glycol and hexamethylene diamines. Polyesteramides are marketed, for example, under the trade name BAK™ from Bayer, such as BAK™ 1095 or BAK™ 2195 for example.

A further group of polymers, which can be used in combination with the aliphatic-aromatic polyester in the polymer composition, are polysaccharides. Polysaccharides are macromolecules in which a relatively large number of sugar residues are glycosidically linked to one another. Suitable polysaccharides in accordance with the invention are polysaccharides having a solubility in dichloromethane at 25° C. of at least 50 g/L. In the context of the invention, polysaccharides also include derivatives thereof if they have a solubility in dichloromethane at 25° C. of at least 50 g/L.

Suitable polysaccharides in accordance with the invention are preferably selected from the group consisting of modified starches such as, in particular, starch ethers and esters, cellulose derivatives such as, in particular, cellulose esters and cellulose ethers, chitin derivatives and chitosan derivatives.

Cellulose derivatives generally refer to celluloses chemically modified by polymer-analogous reactions. They comprise both products in which exclusively the hydroxyl hydrogen atoms of the glucose units of the cellulose have been substituted by organic or inorganic groups and those in which formally an exchange of the entire hydroxyl group has been effected (e.g. desoxycelluloses). Also products which are obtained from intramolecular elimination of water (anhydrocelluloses), oxidation reactions (aldehyde-, keto- and carboxycelluloses) or cleavage of the C₂,C₃-carbon bond of the glucose units (dialdehyde- and dicarboxycelluloses) are counted as cellulose derivatives. Finally, cellulose derivatives are also accessible by reactions such as crosslinking or graft copolymerization reactions. Since for all these reactions to some extent a multiplicity of reagents can be used and, in addition, the degree of substitution and polymerization of the cellulose derivatives obtained can be varied, an extensive palette of soluble and insoluble cellulose derivatives having markedly differing properties is known.

Suitable cellulose ethers are, for example, methyl cellulose, ethyl cellulose, hydroxyethyl cellulose, hydroxypropyl cellulose and hydroxypropylmethyl cellulose.

Suitable cellulose ethers are methylhydroxy-(C₁-C₄)-alkylcelluloses. Methylhydroxy(C₁-C₄)alkyl celluloses are understood to mean methyl hydroxy(C₁-C₄)alkyl celluloses of a wide variety of degrees of methylation and also degrees of alkoxylation.

The preferred methyl hydroxy(C₁-C₄)alkyl celluloses have an average degree of substitution DS of 1.1 to 2.5 and a molar degree of substitution MS of 0.03 to 0.9.

Suitable methylhydroxy(C₁-C₄)alkyl celluloses are for example methyl hydroxyethyl cellulose or methylhydroxypropyl cellulose.

Suitable cellulose esters are, for example, the esters of cellulose with C₂-C₄ monocarboxylic acids, such as cellulose acetate (commercially available from Eastmann CA-398-3), cellulose butyrate, cellulose acetobutyrate, cellulose propionate and cellulose acetopropionate. Cellulose esters are obtainable in a wide variety of degrees of polymerization and substitution.

A further group of polymers, which can be used in combination with the aliphatic-aromatic polyester in the polymer composition, are proteins. Proteins to be used in accordance with the invention comprise polypeptides (acid amide-like condensation products of amino acids linked by peptide bonds) and derivatives thereof having a solubility in dichloromethane at 25° C. of at least 50 g/l. They polypeptides may be of natural or synthetic origin.

According to particularly preferred groups of embodiments, the polymer composition comprises a combination comprising or consisting of
i) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester, selected from the group consisting of polyhydroxy fatty acids, including in particular poly-C₆-C₁₂-lactones, polyhydroxy acetic acid, polylactic acid, and aliphatic-aliphatic polyesters, and mixtures thereof, especially from the group consisting of polylactides, aliphatic-aliphatic polyesters, poly-C₆-C₁₂-lactones and mixtures thereof.

According to especially preferred groups of embodiments, the polymer composition comprises a combination comprising or consisting of
i) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
ii) at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

According to another preferred groups of embodiments, the polymer composition comprises a combination comprising or consisting of
i) at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST);
ii) at least one aliphatic-aliphatic polyester which is at least partially crystalline and has a melting point in the range of 50 to 120° C., particularly in the range of 55 to 115° C.; and
iii) at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

According to another preferred groups of embodiments, the polymer composition comprises or consists of at least one aliphatic-aliphatic polyester which is at least partially crystalline and has a melting point in the range of 50 to 120° C., particularly in the range of 55 to 115° C.

Preference is polymer composition essentially consisting of at least one aliphatic-aliphatic polyester, which is at least partially crystalline and has a melting point in the range of 50 to 120° C., particularly in the range of 55 to 115° C., with one or more polymers that are not aliphatic-aromatic polyesters. The proportion of the aliphatic-aliphatic polyester is 30% to 99% by weight, based on the total weight of aliphatic-aliphatic polyester and the polymer that is not an aliphatic-aromatic polyester. Preferably, the proportion of the aliphatic-aliphatic polyester is 30% to 99% by weight, preferably 35% to 95% by weight, further preferably 40% to 90% by weight, based on the total weight of the aliphatic-aliphatic polyester and the further polymer that is not aliphatic-aromatic polyester.

Another preference is polymer composition essentially consisting of at least one aliphatic-aromatic polyester with one or more polymers that are not aliphatic-aromatic polyesters, with a proportion by weight of the aromatic-aliphatic polyester of 30% to 99% by weight, based on the total weight of aliphatic-aromatic polyester and the polymer that is not an aliphatic-aromatic polyester. Preferably, the proportion of the aliphatic-aromatic polyester is 30% to 80% by weight, preferably 35% to 75% by weight, further preferably 40% to 70% by weight, based on the total weight of the aliphatic-aromatic polyester and the further polymer that is not aliphatic-aromatic polyester.

According to particularly preferred groups of embodiments, the polymer composition comprises a combination comprising or consisting of
a) 30 to 80% by weight, preferably 35 to 75% by weight, more preferably 40 to 70% by weight and especially 45 to 70% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
b) 20 to 70% by weight, preferably 25 to 65% by weight, more preferably 30 to 60% by weight and especially 30 to 55% by weight, in each case based on the total mass of the wall material, of at least one aliphatic polyester, selected from the group consisting of polyhydroxy fatty acids, including in particular poly-C₆-C₁₂-lactones, polyhydroxy acetic acid, polylactic acid, and aliphatic-aliphatic polyesters, and mixtures thereof, especially from the group consisting of polylactides, aliphatic-aliphatic polyesters, poly-C₆-C₁₂-lactones and mixtures thereof.

According to especially preferred groups of embodiments, the polymer composition comprises a combination comprising or consisting of
a) 30 to 80% by weight, preferably 35 to 75% by weight, more preferably 40 to 70% by weight and especially 45 to 70% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST); and
b) 20 to 70% by weight, preferably 25 to 65% by weight, more preferably 30 to 60% by weight and especially 30 to 55% by weight, in each case based on the total mass of the wall material, of at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

According to another preferred groups of embodiments, the polymer composition comprises a combination comprising or consisting of
a) 10 to 80% by weight, preferably 15 to 70% by weight, more preferably 25 to 50% by weight, in each case based on the total mass of the wall material, of at least one aliphatic-aromatic polyester, selected from the group consisting of polybutylene azelate-co-butylene terephthalate (PBAzeT), polybutylene brassylate-co-butylene terephthalate (PBBrasT), polybutylene adipate terephthalate (PBAT), polybutylene sebacate terephthalate (PBSeT) and polybutylene succinate terephthalate (PBST);
b) 10 to 80% by weight, preferably 15 to 75% by weight, more preferably 25 to 70% by weight, in each case based on the total mass of the wall material, of at least one aliphatic-aliphatic polyester which is at least partially crystalline and has a melting point in the range of 50 to 120° C., particularly in the range of 55 to 115° C.; and
c) 10 to 80% by weight, preferably 15 to 75% by weight, more preferably 25 to 70% by weight, in each case based on the total mass of the wall material, of at least one aliphatic polyester, selected from the group consisting of polycaprolactones, of polylactic acid (PLA), polylactid glycolid, polybutylene succinate adipate, polybutylene succinate, polybutylene sebacate and polybutylene succinate sebacate.

According to another preferred groups of embodiments, the polymer composition comprises of consists of 30 to 100% by weight, preferably 35 to 99% by weight, more preferably to 98% by weight and especially 45 to 97% by weight, in each case based on the total mass of the wall material, of at least one aliphatic-aliphatic polyester which is at least partially crystalline and has a melting point in the range of 50 to 120° C., particularly in the range of 55 to 115° C.

According to a further preferred group of embodiments, the continuous phase prepared in step i. comprises a combination of at least one aliphatic-aromatic polyester and at least one further polymer, which is not an aliphatic-aromatic polyester, and which is preferably selected from the group consisting of the aforementioned further polymers, which are mentioned as preferred or especially preferred and mixtures thereof, where the combination of these polymers is dissolved in the water-immiscible solvent. In this solution, the mass ratio of the aliphatic-aromatic polyester and the at least one further polymer, which is not an aliphatic-aromatic polyester, is typically in the range of 30:70 to 99:1 or in the range of 30:70 to 80:20, in particular in the range of 35:65 to 75:25 and especially in the range of 40:60 to 70:30 or in the range of 30:70 to 70:30 or in the range of 45:55 to 70:30.

According to a further preferred group of embodiments, the continuous phase prepared in step i. comprises a combination of at least one aliphatic-aliphatic polyester, which is at least partially crystalline and has a melting point in the range of 50 to 120° C., particularly in the range of 55 to 115° C., and at least one further polymer, which is not an aliphatic-aromatic polyester. The combination of these polymers is dissolved in the water-immiscible solvent. In this solution, the mass ratio of the aliphatic-aliphatic polyester and the at least one further polymer, which is not an aliphatic-aromatic polyester, is typically in the range of 30:70 to 99:1 or in the range of 30:70 to 80:20, in particular in the range of 35:65 to 75:25 and especially in the range of 40:60 to 70:30 or in the range of 30:70 to 70:30 or in the range of 45:55 to 70:30.

Preference is given to mixtures of an aliphatic-aromatic polyester with a further polymer which is not an aliphatic-aromatic polyester, especially the mixtures with aliphatic-aliphatic polyesters, in which the melting point of the aliphatic-aromatic polyester is at least 10° C., preferably at least 20° C., above the melting point of the further polymer, or the glass transition temperature of the aliphatic-aromatic polyester is at least 10° C., preferably at least 20° C., above the glass transition temperature of the further polymer. If the further polymer is an amorphous compound, the melting point of the aliphatic-aromatic polyester is at least 10° C., preferably at least 20° C., above the glass transition temperature of the further polymer.

In a preferred embodiment, the preferred aliphatic-aromatic polyesters are characterized by a molecular weight Mn in the range from 1000 to 100 000 g/mol, further preferably in the range from 1000 to 75 000 g/mol, further preferably in the range from 1500 to 50 000 g/mol.

In a preferred embodiment, the preferred aliphatic-aliphatic polyesters, which are at least partially crystalline and have a melting point in the range of 50 to 120° C., particularly in the range of 55 to 115° C., are characterized by a molecular weight Mn in the range of 1000 to 9000 g/mol, particularly in the range of 1500 to 6500 g/mol, as determined from hydroxyl number.

In addition to the polymer, the solution provided in step i. contains at least one aroma chemical, which is present in the solution in dissolved form. In the process of the invention, it is possible to use either one aroma chemical or a mixture of aroma chemicals. This may be a mixture of aroma chemicals from one class or a mixture of aroma chemicals from different classes.

Preferably, the aroma chemical is liquid at 22° C. and 1013 mbar. According to a preferred embodiment, the aroma chemical has a boiling point of at least 90° C., in particular at least 120° C. at 1013 mbar, e.g. in the range of 90° C. to 300° C., in particular 120 to 250° C.

Preferred aroma chemicals are hydrophobic and, especially at 25° C., have a water solubility in deionized water of not more than 1 g/L, in particular not more than 500 mg/L, especially not more than 100 mg/L.

In step i. the aroma chemical may be provided as such. The aroma chemical may also be provided in pre-dissolved, emulsified or suspended form. Suitable solvents for pre-dissolving, emulsifying or suspending the aroma chemical are especially C₁-C₄-alkanols such as methanol, ethanol, n-propanol, isopropanol, n-butanol, 2-butanol, tert-butanol and aliphatic and cycloaliphatic hydrocarbons such as n-pentane, n-hexane, hexane mixtures, n-heptane, cyclohexane, cycloheptane, methylcyclohexane, petroleum ether, white oils, diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl cyclohexane-1,2-dicarboxylate, and dialkyl adipates such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), and dimethyl sulfoxide and mixtures thereof.

The amount of the aroma chemical in the solution is such that the weight ratio of the aroma chemical to organic polymer composition is frequently in the range of 1:50 to 2:1, in particular in the range of 1:20 to 1:1.5 and especially in the range of 1:10 to 1:2. The amount of aroma chemical refers to the aroma chemical as such.

Typically, the concentration of the aroma chemical in the solution provided in step i. is in the range of 0.5 to 200 g/kg, in particular in the range of 1 to 100 g/kg, especially 1.5 to 50 g/kg, based on the total weight of the solution.

Preferred aroma chemicals for loading of the microparticles are selected, for example, from the following compounds:

alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a CIS isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide 3), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styrenyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methylcyclopentadec-5-enone (Muscenone⁹), 15-pentadec-11-enolide and/or (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹⁰), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyloct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), tetrahydro-2-isobutyl-4-methyl-2H-pyran (Dihydrorosenon⁴), 1,4-bis(ethoxymethyl)cyclohexane (Vertofruct⁴), L-isopulegol (1R,2S,5R)-2-isopropenyl-5-methylcyclohexanol, pyranyl acetate (2-isobutyl-4-methyltetrahydropyran-4-yl acetate), nerol ((Z)-2,6-dimethyl-2,6-octadien-8-ol), neryl acetate, 7-methyl-2H-1,5-benzodioxepin-3(4H)-one (Calone¹⁹⁵¹⁵), 3,3,5-trimethylcyclohexyl acetate (preferably with a content of CIS isomers of 70% by weight) or more and 2,5,5-trimethyl-1,2,3,4,4a,5,6,7-octahydronaphthalen-2-ol (Ambrinol S¹), tetrahydro-4-methyl-2-(2-methylpropenyl)-2H-pyran (rose oxide), 4-methyl-2-(2-methylpropyl)oxane or 4-methyl-2-(2-methylpropyl)-2H-pyran (Dihydrorosan⁴), 2-isobutyl-4-methyltetrahydropyran-4-yl acetate, 2,2-dimethylpropane-1,3-diol diacetate (Velberry⁴), mixture of α- and β-santalol (Isobionics® Santalol), esters as disclosed in WO 2020/016421, preferably esters according to the mixture of example 1.1 (Florascone⁴), prenyl acetate (=3-methylbut-2-enyl acetate), isoamyl acetate, dihydromyrcenol (2,6-dimethyloct-7-en-2-ol) and methylheptenone (6-methylhept-5-en-2-one) and mixtures thereof, and also mixtures thereof with one or more other aromas.

In the context of the present invention, the aforementioned aromas or odorants are accordingly preferably combined with mixtures of the invention.

If trade names are specified above, these refer to the following sources:
¹ trade name of Symrise GmbH, Germany;
² trade name of Givaudan AG, Switzerland;
³ trade name of International Flavors & Fragrances Inc., USA;
⁴ trade name of BASF SE;
⁵ trade name of Danisco Seillans S.A., France;
⁹ trade name of Firmenich S.A., Switzerland;
¹⁰ trade name of PFW Aroma Chemicals B.V., the Netherlands.

More particularly, the advantages of the invention are manifested in the case of aroma chemicals that are selected from volatile fragrances and aroma mixtures comprising at least one volatile fragrance. Volatile fragrances are understood to mean fragrances having a high vapor pressure at room temperature. A fragrance is considered to be a volatile fragrance especially when it has the following property: If a droplet of the volatile fragrance is applied to a strip of paper and left to evaporate off under ambient conditions at room temperature (22° C.), its odor is no longer perceptible to an experienced perfumer no longer than 2 hours after application. The volatile fragrances especially include, but are not limited to the following compounds: rose oxide (tetrahydro-4-methyl-2-(2-methylpropenyl)-2H-pyran), 4-methyl-2-(2-methylpropyl)oxane or 4-methyl-2-(2-methylpropyl)-2H-pyran (Dihydrorosan®), 1,4-bis(ethoxymethyl)cyclohexane (Vertofruct⁴), 2,2-dimethylpropane-1,3-diol diacetate (Velberry⁴), mixture of α- and β-santalol (Isobionics® Santalol), esters as disclosed in WO 2020/016421, preferably esters according to the mixture of example 1.1 (Florascone⁴), prenyl acetate (=3-methylbut-2-enyl acetate), isoamyl acetate, dihydromyrcenol (2,6-dimethyloct-7-en-2-ol) and methylheptenone (6-methylhept-5-en-2-one). If an aroma mixture comprising at least one volatile fragrance is used as an aroma chemical or component thereof, the proportion of the volatile fragrance is generally at least 1% by weight, especially at least 5% by weight, for example 1% to 99% by weight, especially 5% to 95% by weight, based on the total weight of the aroma chemical mixture used as aroma chemical.

Further odorants or aroma chemicals with which the odorants mentioned can be combined to give an odorant composition can be found, for example, in S. Arctander, Perfume and Flavor Chemicals, Vol. I and II, Montclair, N. J., 1969, Author's edition or K. Bauer, D. Garbe and H. Surburg, Common Fragrance and Flavor Materials, 4th. Ed., Wiley-VCH, Weinheim 2001. Specifically, the following may be mentioned:
extracts from natural raw materials such as essential oils, concretes, absolutes, resins, resinoids, balsams, tinctures, for example
ambra tincture; amyris oil; angelica seed oil; angelica root oil; anise oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; cananga oil; cardamom oil; cascarilla oil; cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oakmoss absolute; elemi oil; estragon oil; eucalyptus citriodora oil; eucalyptus oil; fennel oil; spruce needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; camellia oil blue; camellia oil roman; carrot seed oil; cascarilla oil; pine needle oil; spearmint oil; cumin oil; labdanum oil; labdanum absolute; labdanum resin; lavandin absolute; lavandin oil; lavender absolute; lavender oil; lemon grass oil; lovage oil; lime oil distilled; lime oil pressed; linalool oil; litsea cubeba oil; laurel leaf oil; macis oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; oregano oil; palmarosa oil; patchouli oil; perilla oil; Peruvian balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil Spanish; sandalwood oil; celery seed oil; spike lavender oil; star anise oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiver oil; juniper berry oil; wine yeast oil; vermouth oil; wintergreen oil; ylang oil; hyssop oil; civet absolute; cinnamon leaf oil; cinnamon bark oil; and fractions thereof or ingredients isolated therefrom.

Individual odorants are, for example, those from the group of
the hydrocarbons, for example 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols, for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof, for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof, for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds, for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles, for example 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;
the esters of aliphatic carboxylic acids, for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-ylacetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl 2,4-decadienoate;
methyl 2-octynoate; methyl 2-nonynoate; allyl 2-isoamyloxyacetate; methyl 3,7-dimethyl-2,6-octadienoate; 4-methyl-2-pentyl crotonate;
the acyclic terpene alcohols, for example geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones, for example geranial; neral; citronellal; 7-hydroxy-3,7-dimethyloctanal; 7-methoxy-3,7-dimethyloctanal; 2,6,10-trimethyl-9-undecenal; geranyl acetone; and also the dimethyl and diethyl acetals of geranial, neral, 7-hydroxy-3,7-dimethyloctanal; the cyclic terpene alcohols, for example menthol; isopulegol; alpha-terpineol; terpineol-4; menthan-8-ol; menthan-1-01; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones, for example menthone; isomenthone; 8-mercaptomenthan-3-one; carvone; camphor; fenchone; alpha-ionone; beta-ionone; alpha-n-methylionone; beta-n-methylionone; alpha-isomethylionone; betaisomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols, for example 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols, for example alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trim ethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers, for example cineol; cedryl methyl ether; cyclododecyl methyl ether; 1,1-dimethoxycyclododecane; 1,4-bis(ethoxymethyl)cyclohexane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones, for example 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-ylcyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; cyclohexadec-5-en-1-one; 6,7-dihydro-1,1,2,3,3-pentamethyl-4(5H)-indanone; 8-cyclohexadecen-1-one; 7-cyclohexadecen-1-one; (7/8)-cyclohexadecen-1-one; 9-cycloheptadecen-1-one; cyclopentadecanone; cyclohexadecanone;
the cycloaliphatic aldehydes, for example 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones, for example 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols, for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7a-hexahydro-5- or -6-indeny) propionate; 4,7-methano-3a,4,5,6,7,7a-hexahydro-5- or -6-indenyl isobutyrate; 4,7-methanooctahydro-5- or -6-indenyl acetate;
the esters of cycloaliphatic alcohols, for example 1-cyclohexylethyl crotonate;
the esters of cycloaliphatic carboxylic acids, for example allyl 3-cyclohexylpropionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols, for example benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids, for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers, for example 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
the aromatic and araliphatic aldehydes, for example benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropyl phenyl)propanal; 2-methyl-3-(4-tert-butylphenyl) propanal; 2-methyl-3-(4-isobutylphenyl)propanal; 3-(4-tert-butylphenyl) propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl) propanal;
the aromatic and araliphatic ketones, for example acetophenone; 4-methylacetophenone; 4-methoxyacetophenone; 4-tert-butyl-2,6-di methylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl) ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
the aromatic and araliphatic carboxylic acids and esters thereof, for example benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds, for example 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentanonitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters, for example estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
the heterocyclic compounds, for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones, for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In addition, suitable aroma chemicals are macrocyclic carbaldehyde compounds as described in WO 2016/050836.

Particular preference is also given to mixtures of L-menthol and/or DL-menthol, L-menthone, L-menthyl acetate, or L-isopulegol, which are highly sought-after as analogs or substitutes for what are referred to as synthetic dementholized oils (DMOs). The mixtures of these minty compositions are preferably used in the ratio of L-menthol or DL-menthol 20-40% by weight, L-menthone 20-40% and L-menthyl acetate 0-20%, or in the ratio of 20-40% by weight, L-menthone 20-40% and L-isopulegol 0-20%.

The aforementioned aromas and aroma mixtures can be used as such or in a solvent which in itself is not an aroma. Typical solvents for aromas are especially those having a boiling point at standard pressure above 150° C. and which do not dissolve the wall material, e.g. diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl 1,2-cyclohexanedicarboxylate, and dialkyl adipates, such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), dimethyl sulfoxide and white oils.

The solution may contain small amounts of further ingredients, such as stabilizers. In particular, the solution may contain small amounts of further ingredients which stem from the polymer composition, such as stabilizers, catalysts or other processing aids, and/or the aroma chemical, such as degradation products, stabilizers and organic solvents. In particular, the solution prepared in step i. consists essentially of the polymer composition, the aroma chemical and the volatile organic solvent. In particular, the solution consists to at least 95% by weight, in particular to at least 99% by weight, based on the weight of the solution of the polymer composition, the aroma chemical and the volatile organic solvent. Typically, the total amount of these further compounds will not exceed 20% by weight, in particular 10% by weight, based on the total weight of aroma chemical and polymer in the polymer composition. The concentration of these compounds in the solution is in particular less then 50 g/kg, more particularly less than 10 g/kg of the solution, especially less then 5 g/kg or less than 2 g/kg or less than 1 g/kg of the solution.

The solution can be prepared by any conventional means for preparing solution of polymers and organic compounds in organic solvents. For this, the polymer composition, the aroma chemical and the volatile organic solvent are mixed in any order, using a suitable mixing device. Typically, mixing is carried out in a mixing vessel, such as a stirred tank. The temperature for dissolving the polymer composition and the aroma chemical in the volatile organic solvent is not particularly critical. Dissolution may be carried out at room temperature or at any other suitable temperature, e.g. in the range of 0 to 100° C. or higher to achieve a quicker dissolution, in particular of the polymer composition. Dissolution may be carried out under pressure, if the dissolution temperature is higher than the boiling point of the volatile organic solvent is lower than the dissolution temperature.

Process step ii.

Next, this solution of the polymer composition and the aroma chemical is emulsified in an aqueous medium containing at least one dispersant. Thereby, an oil-in-water-emulsion, hereinafter also termed o/w-emulsion, of the solution of the polymer composition and the aroma chemical in the volatile organic solvent is formed. In this o/w emulsion the solution, which contains the polymer composition and the aroma chemical dissolved in the volatile organic solvent, form the disperse phase, while the aqueous phase is the continuous phase.

The aqueous phase contains at least one dispersant in order to stabilize the droplets of the o/w emulsion during its production in step ii., but also during the removal of the volatile organic solvent in step iii.. Dispersants suitable for stabilizing o/w emulsions are common knowledge and are mentioned for example in EP 2794085 and EP 3007815, the teaching of which is expressly incorporated by reference.

Suitable dispersants are typically water soluble polymers, for example, cellulose derivatives such as hydroxyethyl cellulose, methyl hydroxyethyl cellulose, methyl cellulose and carboxymethyl cellulose, polyvinylpyrrolidone, copolymers of vinylpyrrolidone, gelatin, gum arabic, xanthan, casein, polyethylene glycols, and partly or completely hydrolyzed polyvinyl acetates (polyvinyl alcohols), methyl hydroxypropyl cellulose, lignin sulfonates and also mixtures of the above. Inorganic pickering systems, such as colloidal silica and colloidal clay minerals may also be used as dispersants for this purpose.

Preferred dispersants are partly or completely hydrolysed polyvinyl acetates (polyvinyl alcohols) and also methyl hydroxy(C₁-C₄)alkyl celluloses as well as mixtures thereof.

Particular preference is given to partially hydrolysed polyvinyl acetates, also termed partially hydrolysed polyvinyl alcohols (PVAs) with particularly having a degree of hydrolysis of 70% to 99.9%, in particular 75 to 99%, more particular 80 to 95%. In addition, PVA copolymers, as described in WO 2015/165836, are also suitable. The PVA may be in particular a carboxy-modified anionic PVA. Such a carboxy-modified PVA preferably has a proportion of carboxyl groups of 1 to 6 mol %. In particular, a carboxy modified PVA is used as a dispersant, whose 4% by weight aqueous solution preferably has a viscosity in the range of 20.0 to 30.0 mPa*s at 20° C.

Methyl hydroxy(C₁-C₄)alkyl celluloses are understood to mean methyl hydroxy(C₁-C₄)alkyl celluloses of a wide variety of degrees of methylation and also degrees of alkoxylation. The preferred methyl hydroxy(C₁-C₄)alkyl celluloses have an average degree of substitution DS of 1.1 to 2.5 and a molar degree of substitution MS of 0.03 to 0.9. Suitable methyl hydroxy(C₁-C₄)alkyl celluloses are for example methyl hydroxyethyl cellulose or methyl hydroxypropyl cellulose.

A particularly preferred dispersant is methyl hydroxypropyl cellulose, whose 2% aqueous solution has a viscosity in the range of 90 to 700 mPa·s, particularly in the range of 100 to 600 mPa·s, especially in the range of 400 to 550 mPa·s, as determined by Brookfield RVT at 20° C. at 20 rpm on bone-dry basis.

Particularly preferred is a methyl hydroxypropyl cellulose, whose 2% aqueous solution has a viscosity in the range of 400 to 550 mPa·s, as determined by Brookfield RVT at at 20 rpm on bone-dry basis. This methyl hydroxypropyl cellulose is commercially available e.g. as Culmina) MHPC 400 R of Ashland.

Non-limiting examples of commercially available methyl hydroxypropyl cellulose having a viscosity in the above-mentioned ranges are, for example, Culminal MHPC 100, Culminal MHPC 400 R, Culminal MHPC 500 RF of Ashland.

A further particularly preferred dispersant is selected from the group of partially hydrolysed polyvinyl alcohols with particular preference given to those having a degree of hydrolysis of 70% to 99.9%, in particular 75 to 99%, more particular 80 to 95% and especially 85 to 90%. An especially preferred dispersant is a carboxy modified anionic PVA having 1 to 6 mol-% of carboxyl groups, based on the amount of repeating units and a degree of hydrolysis of in the range of 75 to 99%, in particular 80 to 95% and especially 85% to 90%. Amongst these, those are preferred, whose 4% by weight aqueous solution has a viscosity of 20.0 to 30.0 mPa*s at 20° C.

Also preferred dispersants are salts of lignin based sulfonic acids, also termed lignin sulfonates or lignosulfonates. Suitable lignin sulfonates may comprise, for example, sodium lignosulfonate, calcium lignosulfonate, ammonium lignosulfonate, magnesium lignosulfonate, potassium lignosulfonate, or sulfomethylated lignosulfonate.

The suitable lignin based sulfonic acids have an average molar weight Mw of at least 5,000 Da. Preferably, an average molar weight Mw in the range of 5,000 Da to 100,000 Da, as determined by gel permeation chromatography according to DIN 55672-3.

Preferably, said lignin based sulfonic acids have a degree of sulfonation from 1.0 to 2.5 mol per kilogram of said lignosulfonic acid, wherein the degree of sulfonation said lignin based sulfonic acid as applied herein is calculated from the sulfur content of said lignin based sulfonic acid as determined by atomic emission spectroscopy, from which the content of sulfate (determined according to DIN 38405-D5-2) is being subtracted.

Preferred lignin based sulfonic acids are lignosulfonic acid, ethoxylated lignosulfonic acid or oxidized lignins. Particularly preferred lignin sulfonate is a sulfonated kraft lignin, which is commercially available e.g. as Reax® 910 of Ingevity.

Non-limiting examples of commercially available lignin sulfonates include, for example, Greensperse s7, Reax® 85, Reax® 88A, Reax® 907, Reax® 910, Polyfon® o, Hyact, Kraftsperse 25m, and Borresperse NA. Greensperse s7, Kraftsperse 25m and Reax® 85 (commercially available from Ingevity), and Borresperse NA (commercially available from Borregaard AS) comprise sodium lignosulfonate. Reax® 88A, Reax® 907, Reax® 910, Polyfon® o and Hyact (commercially available from Ingevity) comprise sulfonated kraft lignin.

Another group of preferred dispersants are inorganic pickering systems, in particular a colloidal silica.

In the context of this invention, the term “colloidal silica”, also termed colloidal silica dispersion, colloidal nano-particulate silica or a colloidal silica sol, refers to a stable dispersion of amorphous particulate silicon dioxide SiO₂ having particle sizes in the range 3 to 200 nm, preferably in the range of 5 to 170 nm, especially in the range of 10 to 150 nm. In this regard, the particle size of colloidal silica is at least 3 nm, preferably at least 5 nm and even more preferably at least 10 nm. The upper limit is set by the fact that the particles must be able to be present in a stable colloidal silica sol. Consequently, the particle size is at most 200 nm, preferably at most 170 nm and most preferably at most 150 nm, especially at most 100 nm. The particle size of the colloidal silica refers to the volume average particle diameter of the silica particles as determined by static light scattering, as described above.

An example of suitable colloidal silica sol is the colloidal silica sol having a particle size of 9 nm, which is commercially available e.g. under the trade name Bindzil® 30/360 from AkzoNobel.

Another example of suitable colloidal silica sol is the colloidal silica sol having a particle size in the range of 10 to 150 nm, which is commercially available e.g. as Bindzil® from AkzoNobel.

Further suitable silica sols are Bindzil® 15/500, Bindzil® 30/220, Bindzil® 40/200, Bindzil® CC151HS, Bindzil® CC301 (AkzoNobel), Nyacol® 215, Nyacol® 830, Nyacol® 1430, Nyacol® 2034DI as well as Nyacol® DP5820, Nyacol® DP5480, Nyacol® DP5540 etc. (Nyacol Products), Levasil® 100/30, Levasil® 10° F./30, Levasil® 100S/30, Levasil® 200/30, Levasil® 200F/30, Levasil® 300F/30, Levasil® VP 4038, Levasil® VP 4055 (H.C. Starck/Bayer) or also CAB-O-SPERSE® PG 001, CAB-O-SPERSE® PG 002 (aqueous dispersions of CAB-O-SIL®, Cabot), Quartron PL-1, Quartron PL-3 (FusoChemical Co.), Kostrosol 0830, Kostrosol 1030, Kostrosol 1430 (Chemiewerk Bad Kostritz).

In a preferred embodiment, the dispersant is a colloidal silica sol having a particle size in the range of in the range of 3 to 200 nm, particularly in the range of 5 to 170 nm, especially in the range of 10 to 150, which is commercially available e.g. as Bindzil 50/80 of AkzoNobel.

In order to stabilize the o/w emulsion, the dispersant is added to the aqueous phase. The concentration of the dispersant in the aqueous phase is typically in the range from 0.1 to 5.0% by weight, in particular in the range from 0.2 to 4.0% by weight and especially in the range from 0.3 to 2.0% by weight, based on the total weight of the aqueous phase.

With regard of the amount of the solution or step i. to be emulsified, the concentration of dispersant and the relative amount of solution of step i. to aqueous phase is preferably chosen such that the amount of the dispersant is in the range of 0.1 to 10% by weight, in particular in the range of 0.2 to 5% by weight, based on the total weight of the solution of step i. With regard to the polymer composition and the aroma chemical contained in the solution of step i. the amount of dispersant is preferably in the range of 2 to 30% by weight, in partiuclar in the range of 3 to 20% by weight, based on the total weight of the polymer composition and the aroma chemical contained in the solution obtained in step i.

The weight ratio of the solution prepared in step i. to the aqueous phase containing the dispersant is typically in the range from 1:5 to 1:1, in particular in the range of 1:3 to 1:1, and especially in the range of 1:2.5 to 1:1 or 1:2 to 1:1.

To prepare the o/w emulsion in step ii. and for stabilization thereof one or more emulsifiers can be used together with the aforementioned dispersants. In contrast to dispersants, emulsifiers have typically a lower weight of generally not more than 500 g/mol (number average). Preference is given to emulsifiers having an HLB value according to Griffin in the range of 2 to 10, especially in the range of 3 to 8. The HLB value (HLB=hydrophilic lipophilic balance) according to Griffin (W. C. Griffin: Classification of surface-active agents by HLB. In: J. Soc. Cosmet. Chem. 1, 1949, pp. 311-326) is a dimensionless number between 0 and 20 which provides information on the water and oil solubility of a compound. Preferably, these are non-ionic emulsifiers having an HLB value according to Griffin in the range of 2 to 10, particularly in the range of 3 to 8. However, also suitable are anionic and zwitterionic emulsifiers having an HLB value according to Griffin in the range of 2 to 10, particularly in the range of 3 to 8. Such emulsifiers are generally used in an amount from 0.1 to 10% by weight, especially 0.5 to 5% by weight, based on the total weight of the emulsion prepared in step ii. In general, the emulsifier or emulsifiers can be added to the solution of the polymer composition and the aroma chemical in the volatile organic solvent before emulsifying or in the aqueous medium.

Examples of suitable emulsifiers having an HLB value according to Griffin in the range of 2 to 10 are:
sorbitan fatty acid esters, particularly sorbitan mono-, di- and trifatty acid esters and mixtures thereof, such as sorbitan monostearate, sorbitan monooleate, sorbitan monolaurate, sorbitant tristearate, sorbitan sesquioleate, sorbitan dioleate, sorbitan trioleate;
fatty acid esters of glycerol or of polyglycerol such as glycerol monostearate, glycerol distearate, glycerol monooleate, glycerol dioleate, glycerol monostearate monoacetate, glycerol monoacetate monooleate, polyglycerol polyrinoleate (E476), e.g. the commercially available emulsfier PGPR 90;
lactyl esters of fatty acid monoesters of glycerol;
lecithins;
ethoxylated castor oils, ethoxylated hydrogenated castor oils with degrees of ethoxylation in the range of 2 to 20;
ethoxylated and/or propoxylated C₁₂-C₂₂-alkanols having degrees of alkoxylation in the range of 2 to 10, e.g. stearyl alcohol ethoxylate having a degree of ethoxylation in the range of 2 to 5, stearyl alcohol ethoxylate-co-propoxylate having degrees of alkoxylation in the range of 2 to 8, isotridecyl ethoxylates having degrees of ethoxylation in the range of 2 to 3 and isotridecyl ethoxylateco-propoxylates with degrees of alkoxylation in the range of 2 to 5;
ethoxylated and/or propoxylated C₄-C₁₆-alkylphenols having degrees of alkoxylation in the range of 2 to 10, e.g. nonylphenol ethoxylate having degrees of ethoxylation in the range of 2 to 5 and octylphenol ethoxylate having degrees of ethoxylation in the range of 2 to 5.

Preferably, the aqueous phase does not contain more than 1% by weight of emulsifiers, based on the aqueous phase. In particular, the amount of emulsifier does not exceed 20% by weight, in particular 10% by weight, based on the total amount of dispersant and emulsifier.

Typically, the aqueous phase is water, which contains the dispersant and optionally the emulsifier. Additionally, the aqueous phase may contain a defoamer. The concentration of the defoamer in the aqueous phase is typically less than 2 g/kg, in particular not more than 1 g/kg, based on the total weight of the aqueous phase. The aqueous phase may contain small amounts of organic solvents, which are miscible with water. Such solvents include in particular those, which have a boiling point at 1013 mbar of below and have a solubility in deionized water at 20° C. and 1013 mbar of at least 100 g/L. The amount of such solvents will generally not exceed 10% by weight, in particular not exceed 5% by weight or 1% by weight, based on the total weight of the aqueous phase.

The emulsification of the solution obtained in step i. in the aqueous medium to give the o/w emulsion in process step ii. can be effected by standard procedures for producing emulsion. Typically, emulsification is achieved stirring or shearing a mixture of the aqueous phase and the solution obtained in step i. or combination of both.

While it is possible to meter an aqueous phase containing the dispersant into the solution obtained in step i. during emulsification of step i., the aqueous phase containing the dispersant is preferably initially charged into a vessel, and the organic solution containing the polymer composition and the aroma chemical is metered into this aqueous phase with mixing of the two liquids, e.g. with stirring or shearing. It is also possible to continuously combine a stream of the aqueous phase and a stream of the solution of step i. in a mixing chamber and continuously remove the o/w-emulsion from the mixing chamber. The mixing chamber may have static or dynamic mixing elements.

Depending on the energy input into the mixture of the aqueous phase and the solution obtained in step i., it is possible to control the droplet size. Furthermore, the type and amount of dispersant described above influences the size of the emulsion droplets in equilibrium. A suitable amount can be chosen by routine.

While stirring will usually produce larger droplets having an average droplet size D[4,3] in the range from 100 to 600 μm average droplet sizes D[4,3] of at most or below 400 μm, in particular at most 300 μm and especially at most 250 μm, e.g. in the range of 0.5 to 400 μm, in particular 1 to 300 μm, especially 2 to 250 μm are achieved by means of apparatuses for generating a high shear field. It is also possible to introduce sufficient shear energy by vigorous stirring that average droplet sizes with D[4,3] values in the range from 0.5 to 400 μm, preferably of 1 to 300 μm and especially 2 to 250 μm are achieved. Should even higher shear energy input be intended, it may be advantageous to use apparatuses for generating a high shear field.

By adjusting the droplet size of the o/w emulsion and droplet size distribution, it is possible to adjust the particle size and particle size distribution of the final microparticles laden with the aroma chemical. In other words, a small average particle size of the microparticles is achieved by providing an o/w emulsion having a small average droplet size and likewise a narrow droplet size distribution of the o/w emulsion will result in a narrow particle size distribution of the obtained microparticles laden with the aroma chemical.

Suitable stirrer types include propeller stirrers, impeller stirrers, disk stirrers, paddle stirrers, anchor stirrers, pitched-blade stirrers, cross-beam stirrers, helical stirrers, screw stirrers and others.

Suitable apparatuses for shearing, i.e. for generating a high shear field, are dispersing machines operating by the rotor-stator principle, i.e. rotor stator mixer, such as toothed ring dispersing machines also termed gear dispersing machines, further colloid mills and disk mills, high-pressure homogenizers, also termed high pressure mixers, and ultrasound homogenizers. High shear may also be achieved by using a dispersing disc or a cross-blade stirrer with one or multiple stages. High shear may also be achieved by passing the mixture through a microfluidic devices. Amongst apparatuses for shearing, preference is given to dispersing machines operating by the rotor-stator principle for generating the shear field, in particular to toothed ring dispersing machines. The diameter of the rotors and stators is typically in the range between 1 cm and 40 cm, depending on machine size and dispersing performance. The speed of rotation of such dispersing machines is generally in the range from 500 to 20 000 rpm, in particular 1000 to 15000 rpm (revolutions per minute), depending on the construction type. Of course, machines with large rotor diameters rotate at the lower end of the rotation speed range, while machines with small rotor diameters are usually operated at the upper end of the rotation speed range. The circumferential speed of the rotor is typically in the range of 5 to 50 m/s. The distance of the rotating parts from the stationary parts of the dispersing tool is generally 0.1 to 3 mm.

As mentioned above, droplet size can be controlled by the shear energy input into the mixture of the aqueous phase and the solution obtained in step i. The shear energy introduced can be directly derived from the power consumption of the apparatus for generating a shear field, taking account of the heat loss. Thus, the shear energy input into the o/w emulsion is preferably 250 to 25 000 watts h/m³ batch size. Particular preference is given to an energy input of 500 to 15 000, especially 800 to 10 000, watts h/m³ batch size, calculated based on the motor current.

In a preferred embodiment, the emulsification is carried out such that the emulsion droplets of the o/w emulsion have an average diameter D[4,3], determined by means of light scattering, of at most or below 400 μm, e.g. in the range of 0.5 to 400 μm, in particular in the range of 1 to 300 μm and especially 2 to 250 μm. For this, the emulsification typically comprises mixing the solution of step i. with the aqueous phase and homogenization of the mixture. Homogenization is typically achieved by subjecting the mixture to high shear using a suitable device as described above. Mixing and homogenization can be carried out successively or simultaneously.

For example, the solution of step i. and the aqueous phase are mixed with stirring and the obtained emulsion is then homogenized as described herein, for example by
treatment of the emulsion with a rotor stator mixer, in particular a toothed-rim mixer;
applying ultrasound to the emulsion;
treatment of the emulsion with a dispersing disc or a cross-blade stirrer with one or multiple stages;
passing the emulsion through a microfluidic device,
passing the emulsion through a high-pressure homogenizer;
   or by combinations thereof.

For example, an emulsion is prepared by mixing the solution of step i. with the aqueous phase with stirring of the mixture, e.g. at a stirring speed of 100 to 1000 rpm over a period of 1 to 30 minutes. Thereby, an o/w is obtained, wherein the average diameter D[4,3] of droplets, determined by means of light scattering, is at most or below 600 μm, e.g. in the range of 100 to <600 μm. The thus obtained emulsion is then subjected to a homogenization as described above. In a particular embodiment, homogenization is achieved by using a dispersing machine operating by the rotor-stator principle, such as toothed ring dispersing machines (also termed high pressure mixers) and ultrasound homogenizers, in particular by using a rotor-stator machine. For example, the homogenization to give the o/w emulsion is effected with a toothed rim dispersing machine at a circumferential speed of the rotor of 5 to 50 m/s or at a rotational speed of 1000 to 15 000 rpm over a period of 1 to 30 minutes.

Mixing and homogenization can also be carried out simultaneously. For example, a stream of the aqueous phase and a stream of the solution of step i. are continuously combined in a mixing chamber, which is a homogenizer, and the o/w-emulsion is continuously removed from the mixing chamber. The above mentioned homogenizers can be used for this purpose and are adapted for continuous operation. For this purpose, principally any of the aforementioned homogenizers can be used. Preference is given to microfluidic devices and high pressure homogenizers.

The emulsification is usually carried out at a temperature in the range from 10 to 80° C. The temperature of the mixture is preferably selected such that it is below the boiling point of the volatile organic solvent. The mixture is preferably kept at a temperature in the range from 20 to 45° C., especially from 20 to 40° C. Typically, the emulsification is carried out at atmospheric pressure or at a pressure above atmospheric pressure, e.g. at pressure up to 2 bar. However, a slight vacuum may be applied, as long as the pressure is not lower than the vapour pressure of the solvent or solvent mixture at the temperature of the emulsification process. Preferably, the vacuum will not be lower than 800 mbar.

Process step iii.

In step iii. the water-immiscible, volatile organic solvent is removed from the o/w-emulsion obtained in step ii. by evaporation. As the volatile organic solvent used for dissolving the polymer composition and the aromachemical has a boiling point lower than the boiling point of the aroma chemical and, typically also lower than that of water, the removal of the solvent is rather selective and the aroma chemical and the polymer composition remain in the emulsion and solidifiy with progressive removal of the volatile organic solvent by evaporation, whereby an aqueous suspension of the microparticles is obtained. In other words, removal of the volatile organic solvent primarily results in the formation of an aqueous suspension of the microparticles laden with the aroma chemical.

It is believed that removal of the volatile organic solvent from the o/w emulsion obtained in step ii. results in a solidification the polymer composition contained in the droplets of the emulsion, which thereby forms solid particles and entraps the aroma chemical contained in the droplets. In other words, the microparticles are presumably formed in the droplets of the o/w emulsion, probably by polymer/solvent phase separation occurring at the surface of the droplets, while the aroma chemical is entrapped in the interior of the thus formed microparticles. In contrast to the polymer microparticles obtained by filling porous microparticles as described in WO 2018/065481 and WO 2019/193094, essentially no pore formation is observed in the walls of the microparticles in the course of the removal of the water-immiscible solvent. Rather, the microparticles of the present invention have a more or less closed surface with no pores or no visible pores. Therefore, the process for the present invention allows for an efficient encapsulation of the aroma chemical by the polymer material of the polymer composition without the need for a sealing step.

In order to achieve an efficient solidifcation of the microparticles, the evaporation of the solvent is carried out until the concentration of organic solvent in the aqueous suspension is less than 1% by weight, in particular less than 0.5% by weight, more particularly less than 0.2% by weight, especially less 0.1% by weight, based on the total weight of the aqueous suspension. Typically, the evaporation of the solvent is carried out until the amount of the volatile organic solvent remaining in the aqueous suspension is less than 5% by weight, in particular less than 2% by weight, especially less than 1% by weight or less than 0.1% by weight, based on the total weight of polymer composition and aroma chemical.

It is beneficial, if the o/w emulsion is agitated during the evaporation of the solvent. Agitation will reduce coalescence of the droplets, which may be in particular critical during the initial phase of the evaporation, when the concentration of the organic solvent is still high. By avoiding coalescence, the amount of large particle aggregates can be reduced. Agitation can be achieved by stirring or any other means, e.g. by shaking or by circulation of the emulsion by means of a pump. For practical reasons, stirring is preferred. With regard to stirring, we refer to the above.

As the organic solvent preferably has a boiling point which is lower than 100° C. at 1013 mbar, in particular of at most 85° C., only a minor amount of the water contained in the o/w emulsion is removed during the evaporation of step iii., while the majority of water remains in the emulsion, which is converted during evaporation in an aqueous suspension of the polymer microparticles laden with the aroma chemical.

Preferably, the evaporation of the organic volatile solvent is carried out at a temperature in the range of 30 to 80° C., in particular in the range of 35 to 60° C. The evaporation may be carried out at atmospheric pressure. However, it is beneficial, if the evaporation is carried out at a pressure below atmospheric pressure, in particular at a pressure of not more than 800 mbar, in particular at a pressure of at most 700 mbar, especially at a pressure of at most 500 mbar. Preferably, the pressure during evaporation is not lower than 10 mbar, in particular not lower than 20 mbar, especially not lower than 50 mbar. Thereby, the amount of water evaporated during the evaporation can be reduced and, consequently, energy consumption can be minimized. In particular, the evaporation of the organic volatile solvent is carried out at a temperature in the range of 30 to 80° C., in particular in the range of 35 to 60° C. and at a pressure in the range of 20 to 800 mbar, in particular in the range of 50 to 500 mbar.

Further process steps:

As described above, step iii. results in an aqueous suspension of the laden polymer particles. For further applications, the aqueous suspension may be used. However, it is also possible to isolate the microparticles from the aqueous suspension obtained in step iii. by conventional techniques. This further step may be carried out by the separation of the laden polymer microparticles from the aqueous phase, e.g. by filtration or by centrifugation, or by evaporating the water of the aqueous suspension, e.g. in a spray-drying equipment. Regardless of which isolation technique is used, the laden polymer microparticles may be dried. “Dried” is understood to mean that the laden microparticles comprise a residual amount of water of ≤5% by weight, preferably ≤1% by weight, based on the microparticles. The drying may for example be carried out in a stream of air and/or by applying a vacuum, optionally in each case with heating. This is accomplished, depending on the size of the microparticles, by means of convective driers such as spray driers, fluidized bed and cyclone driers, contact driers such as pan driers, paddle driers, contact belt driers, vacuum drying cabinet or radiative driers such as infrared rotary tube driers and microwave mixing driers. It may be also be possible to remove residual water present after isolation of the laden polymer particles from the aqueous suspension by rinsing with ethanol or acetone, and/or blowing the microparticles dry with an inert gas such as air, nitrogen or argon. Optionally, for this purpose, pre-dried and/or pre-heated inert gases may also be used. The laden polymer microparticles may also be washed, preferably with aqueous propanediol solution, for example as a 10% by weight solution and optionally dried thereafter.

The laden polymer microparticles:

The laden polymer microparticles obtained by the process are made of a polymeric “wall material” which encloses the aroma chemical. The polymer wall material corresponds to the polymer composition used in step i. The weight ratio of aroma chemical to polymer in the laden polymer microparticles typically corresponds to the relative amounts of polymer composition and aroma chemical dissolved in step i. and is in the range of 1:50 to 2:1, in particular in the range of 1:20 to 1:1 or 1:20 to 1.5:1 and especially in the range of 1:20 to 1:1.5 or 1:10 to 1:1 or 1:10 to 1:2. The amount of aroma chemical in laden microparticles depends from the volume of the cavities in the interior of the microparticles and is usually in the range of 4 to 67% by weight, in particular in the range of 5 to 50% by weight, especially in the range of 9 to 40% by weight, based on the total weight of the laden polymer microparticles. Consequently, the amount of polymer material in the laden polymer microparticles is typically in the range of 33 to 96% by weight, in particular 50 to 95% by weight, especially in the range of 60 to 91% by weight, based on the total weight of the laden polymer microparticles.

The distribution of the aroma chemical in the microparticles is not particularly limited. As mentioned above, the polymer composition forms somehow a closed wall which encloses the aroma chemical in the interior of the microparticle. In the interior of the microparticles the aroma chemical may be present in a single chamber or compartment formed by the polymer of the polymer composition. The microparticle may also contain several compartments filled with the aroma chemical. These compartments may be separated from each other or connected to each other by channels between the compartments or openings in the compartment walls. The chamber wall is naturally formed by the polymers of the polymer composition. The compartments may be completely filled with the aroma chemical or only partially filled and may contain air or a gas phase saturated with the aroma chemical.

The microparticles obtainable by the process of the present invention are typically sphere-shaped particles. The terms sphere shaped and “spherical” are used synonymously and mean that the particles have approximately the shape of a rotational ellipsoid and especially a spherical shape, where, in a particle in particular, the ratio of the longest axis through the centre of the particle to the shortest axis through the centre of the particle does not exceed a value of 3 and is especially in the range from 1:1 to 2:1. While the surface of the microparticles is more or less closed and essentially has no pores or no visible pores, it typically has shallow depressions or shallow craters respectively. Shallow means that the depth of the depression is smaller than the diameter of the recession. Frequently, the number of depressions per microparticle is more than 1 and may be up to 50 or higher, depending on the size of the microparticles and the size of the depression.

As mentioned above, the surface of the laden polymer particles essentially have no visible pores, as can be seen by microphotography of the microparticles. “Essentially having no pores” means that the average number of visible pores (number average) is less than 10 pores per microparticle, in particular at most 5 pores per microparticle, if any. The average number of visible pores can be determined by microscopy, in particular by scanning electron microscopy with at least 1600 fold magnification as described in WO 2018/065481 and WO 2019/193094, respectively. Visible pores are those having a diameter of at least 20 nm. Pore size can be determined by scanning electron microscopy as described in the experimental part and in WO 2018/065481 and WO 2019/193094.

The laden polymer microparticles obtainable by the process of the present invention preferably have an average particle diameter D[4,3] in the range of 0.5 to 400 μm, particularly in the range of 1 to 300 μm and especially in the range of 2 to 200 μm. In a first preferred embodiment, the average particle diameter D[4,3] of the spherical microparticles is in the range of 1 to 100 μm, particularly in the range of 2 to 50 μm, especially in the range of 2 to 30 μm. In a second preferred embodiment, the average particle diameter D[4,3] of the spherical microparticles is in the range of 20 to 400 μm, particularly in the range of 30 to 300 μm and especially in the range of 50 to 250 μm. The values referred herein are determined by static laser light scattering according to ISO 13320:2009.

The polymer microparticles laden with the aroma chemical preferably have a Sauter diameter, i.e. a D[3,2] value, in the range of 0.4 to 300 μm, particularly in the range of 0.8 to 250 μm and especially in the range of 1.5 to 190 μm.

The polymer microparticles laden with the aroma chemical preferably have a D[v, 0.5] value in the range of 0.3 to 280 μm, particularly in the range of 0.4 to 250 μm and especially in the range of 1 to 180 μm.

Surprisingly, the organoleptic profile is not significantly affected by the microencapsulation. In other words, the organoleptic profile of the laden microcapsules largely corresponds to that of the non-encapsulated aroma chemical and does not change significantly over time.

The present invention further provides compositions of the microparticles laden with an aroma chemical, obtainable by the process of the invention. The compositions of the invention preferably contain the aroma chemical in a total amount of 2 to 70% by weight, in particular 5 to 60% by weight, based on the total weight of the microparticles laden with the aroma chemical, i.e. of the constituents of the composition other than solvents. The constituents of the microparticles, i.e. the constituents of the composition other than solvents, are essentially the aroma chemical and the polymer that forms the wall material and any auxiliaries that are used in the production of the microparticles and are not removed. The compositions of the invention may be in the form either of a suspension or of a powder, preference being given to powders.

The present invention further provides products comprising a composition of the invention. Preference is given to products comprising the compositions of the invention in a proportion by weight of 0.01% to 80% by weight, based on the total weight of the product.

The nature of the product is naturally guided by the nature of the aroma chemical and may be a product which typically comprises, for example, a perfume, a washing product, a cleaning product, a cosmetic product, a personal care product, a hygiene article, a food, a food supplement or a fragrance dispenser.

The present invention further provides for the use of compositions of the invention in the aforementioned products. Preference is given to the use of compositions of the invention in a product selected from perfumes, washing products, cleaning products, cosmetic products, personal care products, hygiene articles, foods, food supplements, fragrance dispensers and fragrances.

Compositions of the invention that comprise a fragrance as aroma chemical may be used in the production of perfumed articles. The olfactory properties and also the physical properties and the non-toxicity of the compositions of the invention underline their particular suitability for the end uses mentioned.

The use of the compositions is found to be particularly advantageous in conjunction with top notes of compositions, by way of example in perfume compositions comprising dihydrorosan, rose oxides or other readily volatile fragrances, e.g. isoamyl acetate, prenyl acetate or methylheptenone. In this case, the release of the important, sought-after top notes is effectively delayed. The fragrance or aroma compositions are accordingly metered in at the suitable point in the requisite amount. In the mint compositions of L-menthol, DL-menthol, L-menthone and L-menthyl acetate described, aside from the aroma effect, a cooling effect is additionally applied in a targeted manner, e.g. in chewing gums, confectionery, cosmetic products, and industrial applications such as in textiles or superabsorbents. A further advantage lies in the high material compatibility of the compositions of the invention even with reactive or comparatively unstable components such as aldehydes, esters, pyrans/ethers, which may exhibit secondary reactions on the surfaces.

The positive properties contribute to use of the compositions of the invention with particular preference in perfume products, personal care products, hygiene articles, textile detergents and in cleaning products for solid surfaces.

The perfumed article is selected, for example, from perfume products, personal care products, hygiene articles, textile detergents and cleaning products for solid surfaces. Preferred perfumed articles of the invention are also selected from:
perfume products selected from perfume extracts, eau de parfums, eau de toilettes, eau de colognes, eau de solide, extrait partum, air fresheners in liquid form, gel form or a form applied to a solid carrier, aerosol sprays, scented cleaners and scented oils;
personal care products selected from aftershaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, for example skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products, for example hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants, for example underarm sprays, roll-ons, deodorant sticks, deodorant creams, products of decorative cosmetics, for example eye shadows, nail varnishes, make-ups, lipsticks, mascara, toothpaste, dental floss;
hygiene articles selected from candles, lamp oils, joss sticks, propellants, rust removers, perfumed freshening wipes, armpit pads, baby diapers, sanitary towels, toilet paper, cosmetic wipes, pocket tissues, dishwasher deodorizer;
cleaning products for solid surfaces selected from perfumed acidic, alkaline and neutral cleaning products, for example floor cleaners, window cleaners, dish-washing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners;
textile detergents selected from liquid detergents, powder detergents, laundry pretreatments such as bleaches, soaking agents and stain removers, fabric softeners, washing soaps, washing tablets.

In a further aspect, the compositions of the invention are suitable for use in surfactant-containing perfumed articles. This is because there is frequently a search—especially for the perfuming of surfactant-containing formulations, for example cleaning products (in particular dishwashing compositions and all-purpose cleaners)—for odorants and/or odorant compositions with a rose top note and marked naturalness.

In a further aspect, the compositions of the invention can be used as products for providing (a) hair or (b) textile fibres with an attractive odour note.

The compositions of the invention are therefore of particularly good suitability for use in surfactant-containing perfumed articles.

It is preferred if the perfumed article is one of the following:
an acidic, alkaline or neutral cleaning product selected in particular from the group consisting of all-purpose cleaners, floor cleaners, window cleaners, dish-washing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish selected in particular from the group consisting of furniture polishes, floor waxes and shoe creams, or
a personal care product selected in particular from the group consisting of shower gels and shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks, deodorant creams, products of decorative cosmetics.

The customary ingredients with which odorants used in accordance with the invention, or odorant compositions of the invention, may be combined are common knowledge and are described for example in WO 2016/050836, the teaching of which is hereby expressly incorporated by reference.

Preference is likewise given to the use of compositions of the invention for controlled release of actives such as crop protecting agents and pharmaceutical agents.

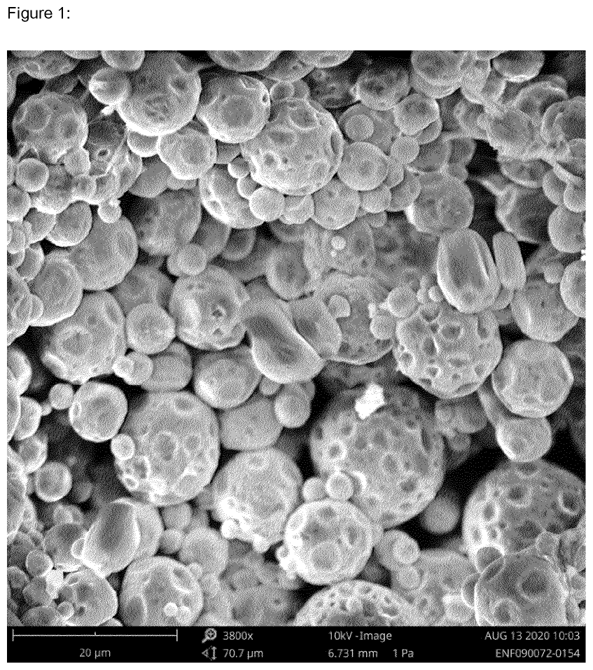

FIG. 1: Microphotography obtained by scanning electron microscopy of the laden microparticles of example 1 after storage for 20 days at ambient temperature at 3800-fold magnification.

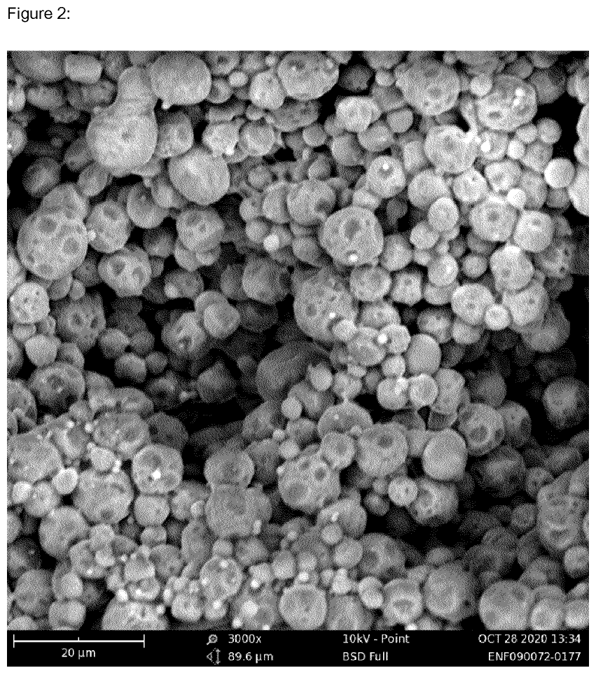

FIG. 2: Microphotography obtained by scanning electron microscopy of the freshly prepared laden microparticles of example 2 at 3000-fold magnification.

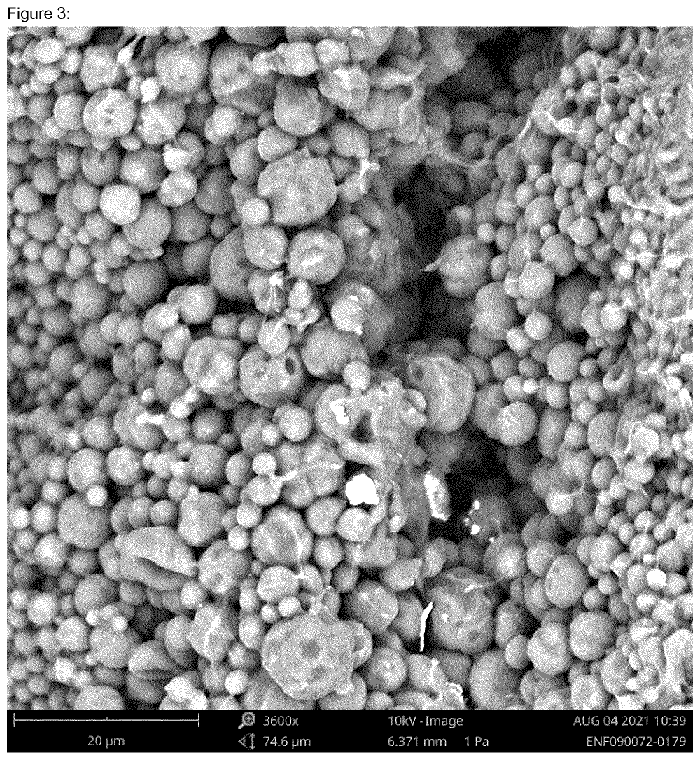

FIG. 3: Microphotography obtained by scanning electron microscopy of the freshly prepared laden microparticles of example 3 at 3600-fold magnification.

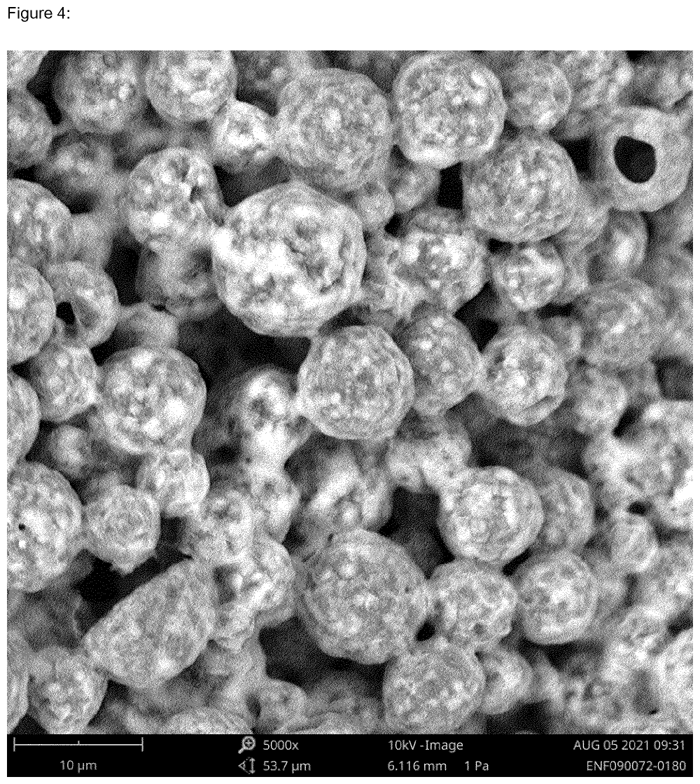

FIG. 4: Microphotography obtained by scanning electron microscopy of the freshly prepared laden microparticles of example 4 at 5000-fold magnification.

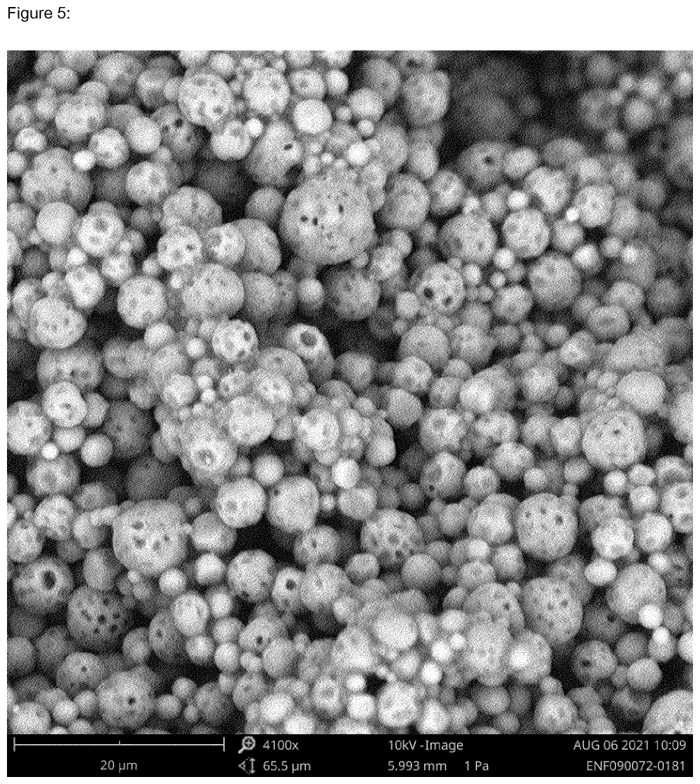

FIG. 5: Microphotography obtained by scanning electron microscopy of the freshly prepared laden microparticles of example 5 at 4100-fold magnification.

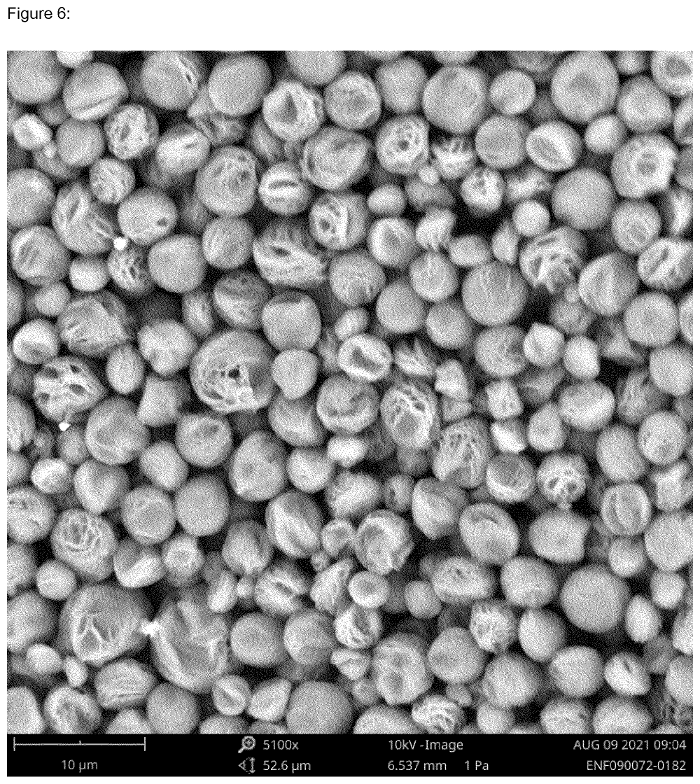

FIG. 6: Microphotography obtained by scanning electron microscopy of the freshly prepared laden microparticles of example 6 at 5100-fold magnification.

### EXAMPLES

Materials

Unless stated otherwise, the following materials and components were used:
Polybutylene sebacate terephthalate (PBSeT): Ecoflex™ FS Blend A1300, melting point in the range of 100-140° C., glass transition temperature of −33° C. (BASF SE);
Polycaprolactone (PCL) having a hydroxyl number of 2 mg KOH/g and a melting point in the range of 58 to 60° C.: product Capa6506 of Perstorp;
Semicrystalline copolyester having a hydroxyl number of 27 to 34 mg KOH/g, a melting point of 65° C., as determined by DSC, and a softening point of 73° C., as determined according to DIN EN ISO 4625:2020-11: Dynacoll® 7381 of Evonik industries;
Polyvinylalcohol: degree of hydrolysis of 88 mol %, a viscosity of a 4% by weight aqueous solution at 20° C. of 25 mPa*s and proportion of carboxyl groups of 3 mol %;
50 wt. % colloidal silica dispersion in water: Bindzil 50/80 of AkzoNobel;
Methylhydroxypropylcellulose: Culminal MHPC 400 R of Ashland;
Medium sulfonated kraft lignin dispersant: Reax® 910 of Ingevity;
defoamer: combination of modified non-ionic fats and hydrophobic silica in
aroma chemical composition: fruity floral odor with woody-spicy note characterized by the following evaporation rate at 30° C. and 1 bar:

TABLE 1

| Time [hours] | Aroma chemical mixture Δ M [%]* |
|---|---|
| 0 | 0 |
| 3 | 5 |
| 5 | 7 |
| 7 | 10 |
| 24 | 22 |
| 48 | 30 |
| 72 | 36 |
| 168 | 41 |
| 336 | 56 |

*Decrease in mass of the aroma chemical mixture in % by weight normalized to the starting value

Methods

Particle diameter: The particle diameter of the o/w emulsion or the particle suspension is determined by static laser light scattering according to ISO 13320:2009 (laser diffraction) with a Malvern Mastersizer 2000 from Malvern Instruments, England, Hydro 2000S sample dispersion unit, by a standard test method documented in the literature.

Scanning electron microscopy: Close-up images were taken from a probe of the microparticles, these were retrospectively automatically measured using the ProSuite (FibreMetric) software from Phenom.

The amount of dichloromethane in the aqueous suspension of microparticles was assessed by determining the chlorine content. The chlorine content was determined by complete incineration of the organic matter and determining the amount of formed hydrochloric acid by coulometric titration.

### Example 1

i. In a glass vessel 5.4 g of the aroma chemical, 6.48 g of PCL and 15.12 g of PBSeT were dissolved at room temperature in 270 g of dichloromethane with stirring until a clear solution of the polymer composition and the aroma chemical in dichloromethane was obtained.
ii. 34.6 g of a 10% by weight aqueous solution of the polyvinyl alcohol, 0.26 g of the defoamer and 388.4 g of deionized water were charged to a 2 L vessel. To this mixture the solution of the polymer composition and the aroma chemical in dichloromethane was added within 30 s with stirring by means of an Ultra Turrax T25 at a rotation speed of 5000 rpm. Then, the mixture was homogenized for 3 minutes by means of the Ultra Turrax at 5000 rpm. The thus obtained o/w emulsion had an average droplet size of less than 15 μm.
iii. Then the Ultra turrax was replaced by an anchor stirrer. A vacuum of 400 mbar was applied, and the vessel was warmed to 45° C. (bath temperature) and solvent was evaporated for 6 h with stirring at 250 rpm. The resulting suspension contained less than <10 ppm dichloromethane.

The mean particle diameter D[v, 0.5] determined from the aqueous suspension was 9.1 μm, the D[v, 0.1] was 1.4 μm and the D[v, 0.9] was 20 μm.

FIG. 1 shows a microphotography of the laden microparticles of example 1 after isolation from the suspension by centrifugation and storage for 20 days at ambient temperature which was obtained by scanning electron microscopy (SEM) of the microparticles at 3800-fold magnification. The microphotography shows that the microparticles have an almost spherical shape with shallow depressions on their surfaces but with no visible pores.

### Example 2

The process was carried out as described for example 1 except for the homogenization, which was carried out at 10.000 rpm for 3 minutes.

The mean particle diameter D[v, 0.5] determined from the thus obtained aqueous suspension was 3.5 μm, the D[v, 0.1] was 1.1 μm and the D[v, 0.9] was 8.9 μm.

FIG. 2 shows a microphotography of the freshly prepared laden microparticles of example 2 after their isolation from the suspension by centrifugation. The microphotography was obtained by scanning electron microscopy of the microparticles at 3000-fold magnification. The microphotography shows that the microparticles have an almost spherical shape with shallow depressions on their surfaces but with no visible pores.

### Example 3

The process was carried out as described for example 2 except for that no defoamer was used.

The mean particle diameter D[v, 0.5] determined from the aqueous suspension was 2.9 μm, the D[v, 0.1] was 1.1 μm and the D[v, 0.9] was 8.3 μm.

FIG. 3 shows a microphotography of the freshly prepared laden microparticles of example 3 after their isolation from the suspension by centrifugation. The microphotography was obtained by scanning electron microscopy of the microparticles at 3600-fold magnification. The microphotography shows that the microparticles have an almost spherical shape with shallow depressions on their surfaces but with no visible pores.

### Example 4

The process was carried out as described for example 2 except for that an alternative dispersant 1 (Blindzil 50/80 and culminal M HPC 400 R) was used in the step ii) instead of polyvinyl alcohol and no defoamer was used. Thereby, pH was adjusted by addition of citric acid to improve the efficiency of the alternative dispersant system. Thus, the step ii) of example 4 is carried out as follows:

ii. 8.1 g of a 50% by weight aqueous solution of the Blindzil 50/80, 0.38 g of a 5% by weight aqueous solution of culminal M HPC 400 R and 415.95 g of deionized water were charged to a 2 L vessel. The pH of the water phase was set to 2.5 by addition of citric acid solution. To this mixture the solution of the polymer composition and the aroma chemical in dichloromethane was added within 30 s with stirring by means of an Ultra Turrax T25 at a rotation speed of 10000 rpm. Then, the mixture was homogenized for 3 minutes by means of the Ultra Turrax at 10000 rpm. The thus obtained o/w emulsion had an average droplet size of less than 15 μm.

The mean particle diameter D[v, 0.5] determined from the aqueous suspension was 8.1 μm, the D[v, 0.1] was 1.9 μm and the D[v, 0.9] was 12.7 μm.

FIG. 4 shows a microphotography of the freshly prepared laden microparticles of example 4 after their isolation from the suspension by centrifugation. The microphotography was obtained by scanning electron microscopy of the microparticles at 5000-fold magnification. The microphotography shows that the microparticles have an almost spherical shape with shallow depressions on their surfaces but with almost no visible pores.

### Example 5

The process was carried out as described for example 2 except for that another alternative stabilizer 2 (Reax 910) was used in the step ii) instead of polyvinyl alcohol and no defoamer was used. Thus, the step ii) of example 5 is carried out as follows: ii. 1.35 g of Reax 910 was dissolved in 416.6 g of deionized water and the solution was charged to a 2 L vessel. To this mixture the solution of the polymer composition and the aroma chemical in dichloromethane was added within 30 s with stirring by means of an Ultra Turrax T25 at a rotation speed of 10000 rpm. Then, the mixture was homogenized for 3 minutes by means of the Ultra Turrax at 10000 rpm. The thus obtained o/w emulsion had an average droplet size of less than 15 μm.

The mean particle diameter D[v, 0.5] determined from the aqueous suspension was 3.3 μm, the D[v, 0.1] was 1.1 μm and the D[v, 0.9] was 10 μm.

FIG. 5 shows a microphotography of the freshly prepared laden microparticles of example after their isolation from the suspension by centrifugation. The microphotography was obtained by scanning electron microscopy of the microparticles at 4100-fold magnification. The microphotography shows that the microparticles have an almost spherical shape with shallow depressions on their surfaces but with almost no visible pores.

### Example 6

The process was carried out as described for example 2 except for that an alternative polymer system (Dynacoll® 7381) was used in the step i) instead of PCL and PBSeT, and no defoamer was used. Thus, the step i) of example 1 is carried out as follows:

i. In a glass vessel 5.4 g of the aroma chemical, 21.6 g Dynacoll® 7381 were dissolved at room temperature in 270 g of dichloromethane with stirring until a clear solution of the polymer composition and the aroma chemical in dichloromethane was obtained.

The mean particle diameter D[v, 0.5] determined from the aqueous suspension was 6 μm, the D[v, 0.1] was 1.3 μm and the D[v, 0.9] was 8.9 μm.

FIG. 6 shows a microphotography of the freshly prepared laden microparticles of example 6 after their isolation from the suspension by centrifugation. The microphotography was obtained by scanning electron microscopy of the microparticles at 5100-fold magnification. The microphotography shows that the microparticles have an almost spherical shape with some shallow depressions on their surfaces but with almost no visible pores.

Following table 2 summarizes important ingredients used in step i) and ii) and results of examples 1 to 6.

TABLE 2

| Step | | Exp. 1 | Exp. 2 | Exp. 3 | Exp. 4 | Exp. 5 | Exp. 6 |
|---|---|---|---|---|---|---|---|
| i) | Polymer | PCL + PBSeT | PCL + PBSeT | PCL + PBSeT | PCL + PBSeT | PCL + PBSeT | Dynacoll ® 7381 |
| | Aroma | aroma chemical | aroma chemical | aroma chemical | aroma chemical | aroma chemical | aroma chemical |
| | chemical | of table 1 | of table 1 | of table 1 | of table 1 | of table 1 | of table 1 |
| ii) | Dispersant | polyvinyl alcohol | polyvinyl alcohol | polyvinyl alcohol | Bindzil 50/80 + | Reax ® 910 | polyvinyl alcohol |
| | | | | | culminal MHPC | | |
| | | | | | 400 R | | |
| | Defoamer | Defoamer given | Defoamer given | No defoamer | No defoamer | No defoamer | No defoamer |
| | | in materials | in materials | | | | |
| Homoge- | No | carried out at | carried out at | carried out at | carried out at | carried out at | carried out at |
| nization | homoge- | 10.000 rpm for | 10.000 rpm for | 10.000 rpm for | 10.000 rpm for | 10.000 rpm for | 10.000 rpm for |
| | nization | 3 min. | 3 min. | 3 min. | 3 min. | 3 min. | 3 min. |

| Result | almost | almost | almost | almost | almost | almost |
|---|---|---|---|---|---|---|
| (SEM) | spherical shape | spherical shape | spherical shape | spherical shape | spherical shape | spherical shape |
| | shallow | shallow | shallow | shallow | shallow | some shallow |
| | depression | depression | depression | depression | depression | depression |
| | no visible pore | no visible pore | no visible pore | no visible pore | no visible pore | no visible pore |

1.-15. (canceled)

16. A process for producing polymer microparticles laden with at least one aroma chemical, which comprises:
i. dissolving the at least one aroma chemical and an organic polymer composition, which contains at least one polyester as a main constituent, in a volatile organic solvent having a boiling point lower than the boiling point of the aroma chemical and having a solubility in water of at most 100 g/L at 20° C. and 1013 mbar, whereby a solution of the aroma chemical and the organic polymer composition is obtained; wherein the concentration of the organic polymer composition in the solution is in the range of 1 to 250 g/kg, based on the total weight of the solution; and
the weight ratio of the aroma chemical to organic polymer composition is in the range of 1:50 to 2:1;
ii. emulsifying the solution obtained in step i. in an aqueous medium containing at least one dispersant;
iii. removing the volatile organic solvent from the emulsion by evaporation at a temperature of below 80° C. and at a pressure below atmospheric pressure, whereby an aqueous suspension of the laden polymer microparticles is obtained.

17. The process of claim 16, where the organic solvent has a boiling point at 1013 mbar in the range of 35 to 85° C.

18. The process of claim 17, where the organic solvent is selected from the group consisting of dichloromethane, trichloromethane, ethyl acetate, benzene, n-hexane, cyclohexane, n-pentane, diethyl ether, methyl tert.-butyl ether, diisopropyl ether and mixtures thereof with 2-butanone.

19. The process of claim 16, where the weight ratio of the aroma chemical to organic polymer composition is in the range of 1:20 to 1.5:1 or 1:20 to 1:1.5.

20. The process of claim 16, where the dispersant contained in the aqueous phase is selected from the group consisting of polysaccharides, polyvinyl alcohols, polymers bearing sulfonate groups, polyvinylpyrolidone copolymers of vinylpyrrolidone and inorganic pickering stabilizers.

21. The process of claim 16, where in step ii. the relative weight of the solution obtained in step i. and the aqueous medium is in the range of 1:5 to 1:1.

22. The process of claim 16, where emulsification comprises mixing the solution of step i. with the aqueous phase and homogenization of the mixture.

23. The process of claim 22, wherein the homogenization is carried out until the average droplet size of the emulsion is at most 400 μm.

24. The process of claim 16, where the evaporation of the solvent is carried out until the concentration of the microparticles in the aqueous suspension is in the range of 2 to 30% by weight, based on the total weight of the suspension.

25. The process of claim 16, wherein the polymer composition comprises at least one aliphatic polyester or at least one semi-aromatic polyester or a combination of at least one semi-aromatic polyester with at least one thermoplastic polymer which is not a semi-aromatic polyester.

26. The process of claim 16, which further comprises the isolation of the microparticles from the aqueous suspension.

27. A composition of microparticles laden with an aroma chemical, obtainable by a process according to claim 16.

28. A product comprising a composition according to claim 27 in a proportion of 0.01% to 80% by weight based on the total weight of the product.

29. The use of the composition according to claim 27 as an additive for imparting a scent or a flavour to a product selected from perfumes, washing and cleaning products, cosmetic products, personal care products, hygiene articles, foods, food supplements, fragrance dispensers and fragrances.

30. The use of the composition according to claim 27 for controlled release of aroma chemicals.
